(19) United States
(12) Patent Application Publication
(10) Pub. No.: US 2005/0009809 A1
(43) Pub. Date: Jan. 13, 2005

(54) Acridine derivatives and their use as medicaments

(76) Inventors: Matthias Gerlach, Brachttal (DE); Peter Emig, Bruchkobel (DE); Klaus Paulini, Maintal (DE); Michael Czech, Frankfurt (DE); Tilman Schuster, Frankfurt (DE); Eckhard Gunther, Maintal (DE)

Correspondence Address: GOODWIN PROCTER L.L.P, 103 EISENHOWER PARKWAY, ROSELAND, NJ, 07068, US

(21) Appl. No.: 10/879,280

(22) Filed: Jun. 29, 2004

Related U.S. Application Data
(60) Provisional application No. 60/486,525, filed on Jul. 11, 2003.

(51) Int. Cl. A61K 31/551; A61K 31/496; C 07D 4 3/02

(52) U.S. Cl. 514/218; 514/253.03; 540/575; 544/361

(57) ABSTRACT

The invention relates to novel acridine derivatives of the formula 1, to their preparation and to their use as medicaments, in particular for the treatment of tumors.

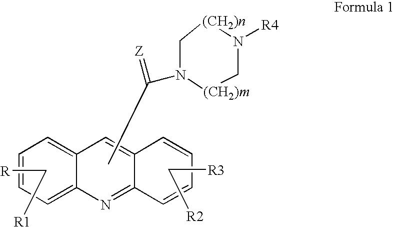

### CROSS-REFERENCE TO RELATED APPLICATION

This application claims the benefit of priority to U.S. Provisional Application No. 60/486,525 filed on Jul. 11, 2003, which is incorporated herein by reference in its entirety.

### BACKGROUND OF THE INVENTION

For the next few years, a dramatic increase in oncoses and tumor-related deaths is expected worldwide. In 2001, worldwide approximately 10 million people were suffering from cancer and over 6 million people died from this disease. The development of tumors is a fundamental disease of higher organisms in the plant kingdom, in the animal kingdom and in humans. The generally recognized multistep model of carcinogenesis assumes that as a result of the accumulation of a number of mutations in an individual cell it is so modified in its proliferation and differentiation behavior that finally, via benign intermediate stages, a malignant state with metastasis is reached. Behind the term cancer or tumor, a clinical picture with more than 200 various individual diseases hides itself. Oncoses can proceed in a benign or malignant manner. The most important tumors are those of the lung, the breast, the stomach, the neck of the uterus, the prostate, the head and neck, the large and small intestine, the liver and the blood system. There are great differences with respect to course, prognosis and therapy behavior. More than 90% of the cases recognized relate to solid tumors, which in particular in the advanced stage or on metastasis are treatable with difficulty or untreatable. The three pillars of cancer control are still surgical removal, irradiation and chemotherapy. In spite of great advances it has still not been possible to develop medicaments which bring about a marked prolongation of the survival time or even a complete cure in the widespread solid tumors. It is therefore meaningful to invent novel medicaments for the control of cancer.

### SUMMARY OF THE INVENTION

The present invention relates to novel acridine derivatives, to their preparation and to their use as medicaments, in particular for treating benign and malignant tumors in humans and mammals.

### DETAILED DESCRIPTION OF THE INVENTION

It has now surprisingly been found, that these novel compounds from the group of the aryl- and heteroaryl-substituted piperazinylcarbonyl acridines and their homologs are suitable for preparing medicaments and these in particular are suitable for treating benign and malignant tumors, and that the compounds according to the invention have better solubility in water than the compounds described in the patent WO0208194. According to this aspect, the present application describes novel compounds from the group of the aryl- and heteroaryl-substituted piperazinylcarbonyl acridines of the formula 1 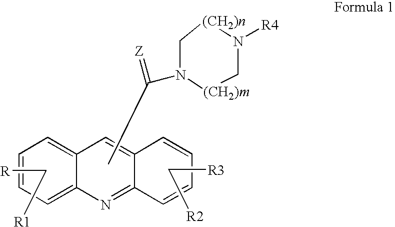
where 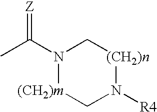
may be attached to carbon atoms C₁ to C₉ of the ring skeleton;
Z: is oxygen or sulfur;
n,m: independently of one another are integers from 0 to 4;
R, R1, R2, R3: may optionally be attached to the heteroaromatic carbon atoms C₁ to C₉ of the acridine, are identical or different and independently of one another are hydrogen, hydroxyl or OR5, but the radicals R, R1, R2 and R3 are not simultaneously hydrogen,
R4: is a (C₆-C₁₄)-aryl radical, a (C₆-C₁₄)-aryl-(C₁-C₄)-alkyl radical or a (C₂-C₁₀)-heteroaryl or (C₂-C₁₀)-heteroaryl-(C₁-C₄)-alkyl radical containing one or more heteroatoms selected from the group consisting of N, O and S, where the (C₁-C₄)-alkyl radical may be unsubstituted or mono- or polysubstituted by identical or different substituents from the group consisting of (C₁-C₆)-alkyl and halogen and the (C₆-C₁₄)-aryl or (C₂-C₁₀)-heteroaryl radical may be unsubstituted or mono- or polysubstituted by identical or different substituents from the group consisting of straight-chain or branched (C₁-C₈)-alkyl, (C₃-C₁₂)-cycloalkyl, straight-chain or branched (C₁-C₈)-alkylcarbonyl, hydroxyl, straight-chain or branched (C₁-C₈)-alkoxy, OR5, halogen, straight-chain or branched aryl-(C₁-C₈)-alkoxy, trityloxy, trimethylsilyloxy, amino, mono-(C₁-C₄)-alkylamino, di-(C₁-C₄)-alkylamino, (C₂-C₅)-cycloalkylamino, morpholino, heterocyclyl-(C₁-C₆)-alkoxy, carboxyl, imidocarboxyl, carboxamidine, straight-chain or branched (C₁-C₈)-alkoxycarbonylamino, straight-chain or branched (C₁-C₈)-alkylcarbonylamino, sulfonyloxy, sulfenyloxy, sulfinyloxy, nitro, nitroso, thiol, straight-chain or branched (C₁-C₈)-alkylthio, straight-chain or branched (C₁-C₈)-alkylsulfonyl, straight-chain or branched (C₁-C₈)-alkylsulfoxy, cyano, isocyanato, straight-chain or branched cyano-(C₁-C₆)-alkyl, straight-chain or branched (C₁-C₈)-alkoxycarbonyl, straight-chain or branched (C₁-C₄)-alkyl which is substituted by one or more halogen atoms, straight-chain or branched carboxy-(C₁-C₈)-alkyl, straight-chain or branched (C₁-C₈)-alkoxycarbonyl-(C₁-C₆)-alkyl, (C₂-C₆)-alkenyl and (C₂-C₆)-alkynyl,
or, if R, R1, R2, R3 may optionally be attached to the heteroaromatic carbon atoms C₁ to C₉ of the acridine, are identical or different and independently of one another:
   are hydrogen, straight-chain or branched (C₁-C₈)-alkyl, (C₃-C₇)-cycloalkyl, straight-chain or branched (C₁-C₈)-alkylcarbonyl, hydroxyl, straight-chain or branched (C₁-C₈)-alkoxy, halogen, straight-chain or branched aryl-(C₁-C₈)-alkoxy, trityloxy, trimethylsilyloxy, amino, mono-(C₁-C₄)-alkylamino, di-(C₁-C₄)-alkylamino, (C₂-C₅)-cycloalkylamino, morpholino, heterocyclyl-(C₁-C₆)-alkoxy, carboxyl, imidocarboxyl, carboxamidine, straight-chain or branched (C₁-C₈)-alkoxycarbonylamino, straight-chain or branched (C₁-C₈)-alkylcarbonylamino, sulfonyloxy, sulfenyloxy, sulfinyloxy, nitro, nitroso, thiol, straight-chain or branched (C₁-C₈)-alkylthio, straight-chain or branched (C₁-C₈)-alkylsulfonyl, straight-chain or branched (C₁-C₈)-alkylsulfoxy, cyano, isocyanato, straight-chain or branched cyano-(C₁-C₆)-alkyl, straight-chain or branched (C₁-C₈)-alkoxycarbonyl, straight-chain or branched (C₁-C₄)-alkyl which is substituted by one or more halogen atoms, straight-chain or branched carboxy-(C₁-C₈)-alkyl, straight-chain or branched (C₁-C₈)-alkoxycarbonyl-(C₁-C₆)-alkyl, (C₂-C₆)-alkenyl, (C₂-C₆)-alkynyl, aryl, where the aryl radical may be unsubstituted or mono- or polysubstituted by identical or different substituents from the group consisting of halogen, straight-chain or branched (C₁-C₈)-alkyl, (C₃-C₁₂)-cycloalkyl, straight-chain or branched (C₁-C₈)-alkylcarbonyl, hydroxyl, straight-chain or branched (C₁-C₈)-alkoxy, amino, mono-(C₁-C₄)-alkylamino, di-(C₁-C₄)-alkylamino, (C₂-C₅)-cycloalkylamino, morpholino, heterocyclyl-(C₁-C₆)-alkoxy, carboxyl, imidocarboxyl, carboxamidine, straight-chain or branched (C₁-C₈)-alkoxycarbonylamino, straight-chain or branched (C₁-C₈)-alkylcarbonylamino, sulfonyloxy, sulfenyloxy, sulfinyloxy, nitro, nitroso, thiol, straight-chain or branched (C₁-C₈)-alkylthio, cyano, isocyanato, straight-chain or branched (C₁-C₈)-alkoxycarbonyl, straight-chain or branched (C₁-C₄)-alkyl, (C₂-C₆)-alkenyl, (C₂-C₆)-alkynyl, substituted by one or more halogen atoms,
R4 is: a (C₆-C₁₄)-aryl radical, a (C₆-C₁₄)-aryl-(C-C₁₄)-alkyl radical or a (C₂-C₁₀)-heteroaryl or (C₂-C₁₀)-heteroaryl-(C₁-C₄)-alkyl radical which contains one or more heteroatoms selected from the group consisting of N, O and S, where the (C₁-C₄)-alkyl radical may be unsubstituted or mono- or polysubstituted by identical or different substituents from the group consisting of (C₁-C₆)-alkyl and halogen and the (C₆-C₁₄)-aryl or (C₂-C₁₀)-heteroaryl radical may be mono- or polysubstituted by identical or different OR5,
and in all cases, R5 may be:
   a sulfone of the formula —SO2-X1, where X1 is NMe₂, hydroxyl, O— alkyl, unsubstituted or substituted alkyl, unsubstituted or substituted cycloalkyl, unsubstituted or substituted aryl, unsubstituted or substituted heteroaryl, unsubstituted or substituted alkylcycloalkyl, unsubstituted or substituted alkylheterocyclyl, unsubstituted or substituted alkylaryl, unsubstituted or substituted alkylheteroaryl;
   —C(O)—X2, where X2 is unsubstituted or substituted alkyl, unsubstituted or substituted cycloalkyl, unsubstituted or substituted heterocyclyl, unsubstituted or substituted aryl, unsubstituted or substituted heteroaryl, unsubstituted or substituted alkylcycloalkyl, unsubstituted or substituted alkylheterocyclyl, unsubstituted or substituted alkylaryl or unsubstituted or substituted alkylheteroaryl,
   —C(O)O—X3, where X3 is unsubstituted or substituted alkyl, unsubstituted or substituted cycloalkyl, unsubstituted or substituted heterocyclyl, unsubstituted or substituted aryl, unsubstituted or substituted heteroaryl, unsubstituted or substituted alkylcycloalkyl, unsubstituted or substituted alkylheterocyclyl, unsubstituted or substituted alkylaryl or unsubstituted or substituted alkylheteroaryl,
   —C(O)NX4X5, where X4 and X5 independently of one another are hydrogen, unsubstituted or substituted alkyl, unsubstituted or substituted cycloalkyl, unsubstituted or substituted heterocyclyl, unsubstituted or substituted aryl, unsubstituted or substituted heteroaryl, unsubstituted or substituted alkylcycloalkyl, unsubstituted or substituted alkylheterocyclyl, unsubstituted or substituted alkylaryl, unsubstituted or substituted alkylheteroaryl, or X4 and X5 together are cycloalkyl or cycloheteroalkyl,
   —P(O)OX6OX7, where X6 and X7 independently of one another are hydrogen, a metal, unsubstituted or substituted alkyl, unsubstituted or substituted cycloalkyl, unsubstituted or substituted heterocyclyl, unsubstituted or substituted aryl, unsubstituted or substituted heteroaryl, unsubstituted or substituted alkylcycloalkyl, unsubstituted or substituted alkylheterocyclyl, unsubstituted or substituted alkylaryl, unsubstituted or substituted alkylheteroaryl, or X6 and X7 together are cycloalkyl or cycloheteroalkyl,
   —P(O)NX8X9NX10X11, where X8, X9, X10 and X11 independently of one another are hydrogen, unsubstituted or substituted alkyl, unsubstituted or substituted cycloalkyl, unsubstituted or substituted heterocyclyl, unsubstituted or substituted aryl, unsubstituted or substituted heteroaryl, unsubstituted or substituted alkylcycloalkyl, unsubstituted or substituted alkylheterocyclyl, unsubstituted or substituted alkylaryl, unsubstituted or substituted alkylheteroaryl, or X8 and X9 or X10 and X11 together are cycloalkyl or cycloheteroalkyl,
   cycloalkyl, alkylcycloalkyl, cycloheteroalkyl or alkylcycloheteroalkyl.

The terms given to explain the compounds of the formula (1) have the following meanings, unless indicated otherwise in the description or in the claims.

The expression “metal” within the meaning of this invention comprises metal ions such as sodium, potassium, lithium, magnesium, calcium, zinc and manganese ions.

The expression “alkyl” within the meaning of this invention comprises acyclic saturated or unsaturated hydrocarbon radicals, which can be branched or straight-chain and unsubstituted or mono- or polysubstituted, having 1 to 20 carbon atoms, i.e. C₁₋₂₀-alkanyls, C₂₋₂₀-alkenyls and C₂₋₂₀-alkynyls. In this context, alkenyls have at least one C—C double bond and alkynyls at least one C—C triple bond. Advantageously, alkyl is selected from the group which comprises methyl, ethyl, n-propyl, 2-propyl, n-butyl, sec-butyl, tert-butyl, n-pentyl, iso-pentyl, neopentyl, n-hexyl, 2-hexyl, n-octyl, ethylenyl (vinyl), ethynyl, propenyl (—CH₂CH═CH₂; —CH═CH—CH₃, —C(═CH₂)—CH₃), propynyl (—CH₂—C≡CH, —C≡C—CH₃), butenyl, butynyl, pentenyl, pentynyl, hexenyl, hexynyl, octenyl and octynyl.

The expression “cycloalkyl” for the purposes of this invention denotes cyclic hydrocarbons having 3-12 carbon atoms, which can be saturated or unsaturated, unsubstituted or substituted. The cycloalkyl radical can also be part of a bi- or polycyclic system.

The expression “heterocyclyl” stands for a 3-, 4-, 5-, 6-, 7- or 8-membered cyclic organic radical, which contains at least 1, optionally 2, 3, 4 or 5 heteroatoms, where the heteroatoms are identical or different and the cyclic radical is saturated or unsaturated, but not aromatic and can be unsubstituted or mono- or polysubstituted. The heterocycle can also be part of a bi- or polycyclic system. Preferred heteroatoms are nitrogen, oxygen and sulfur. It is preferred that the heterocyclyl radical is selected from the group which contains tetrahydrofuryl, tetrahydropyranyl, pyrrolidinyl, piperidinyl, piperazinyl and morpholinyl, where the bonding to the compound of the general formula 1 can take place via any desired ring member of the heterocyclyl radical.

The expression “aryl” within the meaning of this invention means aromatic hydrocarbons, inter alia phenyls, naphthyls and anthracenyls. The radicals can also be fused to further saturated, (partially) unsaturated or aromatic ring systems. Each aryl radical can be present in unsubstituted or mono- or polysubstituted form, where the aryl substituents can be identical or different and can be in any desired and possible position of the aryl.

The expression “heteroaryl” stands for a 5-, 6- or 7-membered cyclic aromatic radical, which contains at least 1, optionally also 2, 3, 4 or 5 heteroatoms, where the heteroatoms are identical or different and the heterocycle can be unsubstituted or mono- or polysubstituted; in the case of substitution on the heterocycle, the heteroaryl substituents can be identical or different and are in any desired and possible position of the heteroaryl. The heterocycle can also be part of a bi- or polycyclic system. Preferred heteroatoms are nitrogen, oxygen and sulfur. It is preferred that the heteroaryl radical is selected from the group which contains pyrrolyl, furyl, thienyl, thiazolyl, triazolyl, tetrazolyl, oxazolyl, isothiazolyl, isoxazolyl, pyrazolyl, imidazolyl, pyridinyl, pyrimidinyl, pyrazinyl, triazinyl, benzothiazolyl, indolyl, indolizinyl, quinolinyl, isoquinolinyl, cinnolinyl, quinazolinyl, quinoxalinyl, phthalazinyl, carbazolyl, phenazinyl, phenothiazinyl, purinyl, acridinyl, phenanthrinyl, where the bonding to the compounds of the general formula 1 can take place via any desired and possible ring member of the heteroaryl radical.

The expressions “alkylcycloalkyl”, “alkylheterocyclyl”, “alkylaryl” or “alkylheteroaryl” mean for the purposes of the present invention that alkyl and cycloalkyl, heterocyclyl, aryl and heteroaryl have the meanings defined above and the cycloalkyl, heterocyclyl, aryl or heteroaryl radical is bonded via a C1-8-alkyl group to the compound of the general formula 1.

In connection with “alkyl”, “alkenyl” and “alkynyl”, the term substituted is understood within the meaning of this invention as meaning the substitution of a hydrogen radical by F, Cl, Br, I, CN, NH₂, NH-alkyl, NH-cycloalkyl, NH-aryl, NH-heteroaryl, NH-alkylaryl, NH-alkylheteroaryl, NH-heterocyclyl, NH-alkyl-OH, N(alkyl)₂, N(alkylaryl)₂, N(alkylheteroaryl)₂, N(heterocyclyl)₂, N(alkyl-OH)₂, NO, NO₂, SH, S-alkyl, S-cycloalkyl, S-aryl, S-heteroaryl, S-alkylaryl, S-alkylheteroaryl, S-heterocyclyl, S-alkyl-OH, S-alkyl-SH, S—S-alkyl, S—S-cycloalkyl, S—S-aryl, S—S-heteroaryl, S—S-alkylaryl, S—S-alkylheteroaryl, S—S-heterocyclyl, S—S-alkyl-OH, S—S-alkyl-SH, S—S-alkyl-C(O)-NH-heterocyclyl, OH, O-alkyl, O-cycloalkyl, O-aryl, O-heteroaryl, O-alkylaryl, O-alkylheteroaryl, O-heterocyclyl, O-alkyl-OH, CHO, C(O)-alkyl, C(S)-alkyl, C(O)-aryl, C(S)-aryl, C(O)-alkylaryl, C(S)-alkylaryl, C(O)-heterocyclyl, C(O)-heteroaryl, C(O)-alkylheteroaryl, C(S)-heterocyclyl, CO₂H, CO₂-alkyl, CO₂-cyclyl, CO₂-heterocyclyl, CO₂-aryl, CO₂-heteroaryl, CO₂alkylaryl, C(O)—NH₂, C(O)NH-alkyl, C(O)NH-aryl, C(O)NH-heterocyclyl, C(O)NH-alkylheterocyclyl, C(O)N(alkyl)₂, C(O)N(alkylaryl)₂, C(O)N(alkylheteroaryl)₂, C(O)N(heterocyclyl)₂, SO-alkyl, SO₂-alkyl, SO₂NH₂, SO₃H, alkyl, cycloalkyl, aryl, heteroaryl, or heterocyclyl, where polysubstituted radicals are to be understood as meaning those which are either polysubstituted, e.g. di- or trisubstituted, on different or on identical atoms, for example trisubstituted on the same C atom as in the case of CF₃, —CH₂CF₃ or in different positions as in the case of —CH(OH)—CH═CH—CHCl₂. Polysubstitution can take place with the same or different substituents.

With respect to aryl, heterocyclyl, heteroaryl, alkylaryl and cycloalkyl, mono- or polysubstituted is understood within the meaning of this invention as meaning the mono- or polysubstitution, e.g. di-, tri- or tetrasubstitution, of one or more hydrogen atoms of the ring system by F, Cl, Br, I, CN, NH₂, NH-alkyl, NH-aryl, NH-heteroaryl, NH-alkylaryl, NH-alkylheteroaryl, NH-heterocyclyl, NH-alkyl-OH, N(alkyl)₂, NC(O)alkyl, N(alkylaryl)₂, N(alkylheteroaryl)₂, N(heterocyclyl)₂, N(alkyl-OH)₂, NO, NO₂, SH, S-alkyl, S-aryl, S-heteroaryl, S-alkylaryl, S-alkylheteroaryl, S-heterocyclyl, S-alkyl-OH, S-alkyl-SH, OH, O-alkyl, O-aryl, O-heteroaryl, O-alkylaryl, O-alkylheteroaryl, O-heterocyclyl, O-alkyl-OH, O-C(O)-alkyl, CHO, C(O)-alkyl, C(S)-alkyl, C(O)-aryl, C(S)-aryl, C(O)-alkylaryl, C(S)-alkylaryl, C(O)-heterocyclyl, C(S)-heterocyclyl, CO₂H, CO₂-alkyl, CO₂-alkylaryl, C(O)—NH₂, C(O)NH-alkyl, C(O)NH-aryl, C(O)NH-heterocyclyl, C(O)N(alkyl)₂, C(O)N(alkylaryl)₂, C(O)N(alkylheteroaryl)₂, C(O)N(heterocyclyl)₂, SO-alkyl, SO₂-alkyl, SO₂-aryl, SO₂-heteroaryl, SO₂NH₂, SO₃H, CF₃, CHO, CHS, alkyl, cycloalkyl, aryl, heteroaryl, and/or heterocyclyl, on one or optionally different atoms (where one substituent can optionally for its part be substituted). Polysubstitution in this case takes place with identical or with different substituents.

If the compounds of the general formula 1 according to the invention have at least one asymmetric center, they can be present in the form of their racemates, in the form of the pure enantiomers and/or diastereomers or in the form of mixtures of these enantiomers and/or diastereomers. The mixtures can be present in any desired mixing ratio of the stereoisomers.

If possible, the compounds according to the invention can be present in the form of the tautomers.

Thus, for example, the compounds according to the invention as in the general formula 1, which have one or more centers of chirality and which occur as racemates, can be separated into their optical isomers, that is enantiomers or diastereomers, by methods known per se. The separation can be carried out by column separation on chiral phases or by recrystallization from an optically active solvent or using an optically active acid or base or by derivatization with an optically active reagent, such as, for example, an optically active alcohol, and subsequent removal of the radical.

The compounds of the general formula 1 according to the invention can, if they have a sufficiently basic group, such as, for example, a secondary or tertiary amine, be converted into salts using inorganic and organic acids. Preferably, the pharmaceutically acceptable salts of the compounds according to the invention as in the general structure 1 with hydrochloric acid, hydrobromic acid, sulfuric acid, phosphoric acid, methanesulfonic acid, p-toluenesulfonic acid, carbonic acid, formic acid, acetic acid, sulfoacetic acid, trifluoroacetic acid, oxalic acid, malonic acid, maleic acid, succinic acid, tartaric acid, racemic acid, malic acid, embonic acid, mandelic acid, fumaric acid, lactic acid, citric acid, taurocholic acid, glutamic acid or aspartic acid are formed. The salts formed are, inter alia, hydrochlorides, hydrobromides, sulfates, phosphates, methanesulfonates, tosylates, carbonates, hydrogencarbonates, formates, acetates, sulfoacetates, triflates, oxalates, malonates, maleates, succinates, tartrates, malates, embonates, mandelates, fumarates, lactates, citrates and glutamates. The stoichiometry of the salts of the compounds according to the invention formed can in this case be an integral or nonintegral multiple of one.

The compounds of the general formula 1 according to the invention can, if they contain a sufficiently acidic group, such as, for example, the carboxyl group, sulfonic acid, phosphoric acid or a phenolic group, be converted into their physiologically tolerable salts with inorganic and organic bases. Possible inorganic bases are, for example, sodium hydroxide, potassium hydroxide, calcium hydroxide, as organic bases ethanolamine, diethanolamine, triethanolamine, cyclohexylamine, dibenzylethylenediamine and lysine. The stoichiometry of the salts of the compounds according to the invention formed can in this context be an integral or nonintegral multiple of one.

Likewise preferred are solvates and in particular hydrates of the compounds according to the invention, which can be obtained, for example, by crystallization from a solvent or from aqueous solution. In this context, one, two, three or as many solvate or water molecules as liked can be combined with the compounds according to the invention to give solvates and hydrates.

It is known that chemical substances form solids which are present in various atomic states, which are described as polymorphic forms or modifications. The different modifications of a polymorphic substance can differ greatly in its physical properties. The compounds of the general formula 1 according to the invention can be present in various polymorphic forms, in this context certain modifications can be metastable.

Most preferred are compounds of the formula I which are found in the following selection:
(1,3-dihydroxyacridin-9-yl)-[4-(6-methylpyridin-2-yl)piperazin-1-yl]methanone (1)
3-[4-(acridin-9-ylcarbonyl)piperazin-1-yl]phenyl isopropylcarbamate (2)
3-[4-(acridin-9-ylcarbonyl)piperazin-1-yl]phenyl acetate (3)
mono-{3-[4-(acridin-9-ylcarbonyl)piperazin-1-yl]phenyl}phosphate (4)
3-[4-(acridin-9-ylcarbonyl)piperazin-1-yl]phenyl methyl carbonate (5)
3-[4-(acridin-9-ylcarbonyl)piperazin-1-yl]phenyl 2-chloroethyl carbonate (6)
3-[4-(acridin-9-ylcarbonyl)piperazin-1-yl]phenyl (2-hydroxyethyl)carbamate (7)
[4-(3-chlorophenyl)piperazin-1-yl]-(1,3-dihydroxyacridin-9-yl)methanone (8)
[4-(6-chloropyridin-2-yl)piperazin-1-yl]-(1,3-dihydroxyacridin-9-yl)methanone (9)
(1,3-dihydroxyacridin-9-yl )-(2,3,5,6-tetrahydro-[1,2′]-bipyrazinyl-4-yl)methanone (10)
bis-{3-[4-(acridin-9-ylcarbonyl)piperazin-1-yl]phenyl}phosphate (11)
(1,3-dihydroxyacridin-9-yl)-[4-(6-methoxypyridin-2-yl)piperazin-1-yl]methanone (12)
(1,3-dihydroxyacridin-9-yl)-[4-(3-hydroxyphenyl)piperazin-1-yl]methanone (13)
(1,3-dihydroxyacridin-9-yl )-[4-(3-methoxyphenyl)piperazin-1-yl]methanone (14)
3-[4-(acridin-9-ylcarbonyl)piperazin-1-yl]phenyl methanesulfonate (15)
3-[4-(acridin-9-ylcarbonyl)piperazin-1-yl]phenyl 2,2-dimethyl-[1,3]-dioxolan-4-ylmethyl carbonate (16)
3-(diphenoxyphosphoryloxy)-9-[4-(3-methoxyphenyl)piperazin-1-yl-carbonyl]acridin-1-yl diphenyl phosphate (17)
3-acetoxy-9-[4-(3-methoxyphenyl)piperazin-1-ylcarbonyl]acridin-1-yl acetate (18)
bis-{3-[4-(acridin-9-ylcarbonyl)piperazin-1-yl]phenyl}carbonate (19)
3-[4-(acridin-9-ylcarbonyl)piperazin-1-yl]phenyl N,N-bis-(2-hydroxyethyl )succinate (20)
3-[4-(acridin-9-ylcarbonyl)piperazin-1-yl]phenyl 4-(4-methylpiperazin-1-yl)-4-oxobutyrate (21)
mono-{3-[4-(acridin-9-ylcarbonyl)piperazin-1-yl]phenyl}glutarate (22)
3-[4-(acridin-9-ylcarbonyl)piperazin-1-y;]phenyl 5-(2,2-dimethyl-[1,3]dioxolan-4-yl)-2,2-dimethyltetrahydrofuro[2,3-d][1,3]dioxol-6-yl succinate (23)
3-[4-(acridin-9-ylcarbonyl)piperazin-1-yl]phenyl 5-[5-(2,2-dimethyl-[1,3]-dioxolan-4-yl)-2,2-dimethyltetrahydrofuro[2,3-d][1,3]dioxol-6-yloxy]-pentanoate (24)
3-[4-(acridin-9-ylcarbonyl)piperazine-1-yl]phenyl 2,3,5-trihydroxy-6-hydroxymethyltetrahydropyran-4-yl succinate (25)
3-[4-(acridin-9-ylcarbonyl)piperazin-1-yl]phenyl 5-(2,3,5-trihydroxy-6-hydroxymethyltetrahydropyran-4-yloxy)pentenoate (26)
3-[4-(acridin-9-ylcarbonyl)piperazin-1-yl]phenyl [1,4′]bispiperidinyl-1′-carboxylate (27)
3-[4-(acridin-9-ylcarbonyl)piperazin-1-yl]phenyl 4-morpholin-4-yl-piperidine-1-carboxylate (28)
3-[4-(acridin-9-ylcarbonyl)piperazin-1-yl]phenyl (2-morpholin-4-ylethyl)carbamate (29)
3-[4-(acridin-9-ylcarbonyl)piperazin-1-yl]phenyl (2-chloroethyl)carbamate (30)
3-[4-(acridin-9-ylcarbonyl)piperazin-1-yl]phenyl diethylcarbamate (31)
bis(dimethylamide) mono-{3-[4-acridin-9-ylcarbonyl)piperazin-1-yl]phenyl}phosphate (32)
mono-{3-[4-(acridin-9-ylcarbonyl)piperazin-1-yl]phenyl}phosphate disodium salt (33)
3-[4-acridin-9-ylcarbonyl)piperazin-1-yl]phenyl nonadecanoate (34)
1-hydroxy-9-[4-(3-methoxyphenyl)piperazin-1-ylcarbonyl]acridin-3-yl diphenyl phosphate (35)
3-hydroxy-9-[4-(3-methoxyphenyl)piperazin-1-ylcarbonyl]acridin-1-yl diphenyl phosphate (36)
(2-hydroxyacridin-9-yl)-[4-(3-methoxyphenyl)piperazin-1-yl]methanone (37)
(2-hydroxyacridin-9-yl)-[4-(3-hydroxyphenyl)piperazin-1-yl]methanone (38)
(1,3-dihydroxyacridin-9-yl)-[4-(4-methyl pyridin-2-yl)piperazin-1-yl]methanone (39)
3-acetoxy-9-[4-(6-methylpyridin-2-yl )piperazin-1-ylcarbonyl]acridin-1-yl acetate (40)
3-acetoxy-9-[4-(6-methoxypyridin-2-yl)piperazin-1-ylcarbonyl]acridin-1-yl acetate (41)

According to a further aspect of the invention, a process for preparing acridine derivatives is provided, which process comprises reacting an acridine carboxylic acid of the formula 2 in which R, R₁, R₂, R₃ are as defined above, Z is an oxygen or sulfur atom and Y is a leaving group, such as halogen, hydroxyl, (C₁-C₆)-alkoxy, preferably methoxy or ethoxy, —O-tosyl, —O-mesyl, tetrazolyl or imidazolyl, 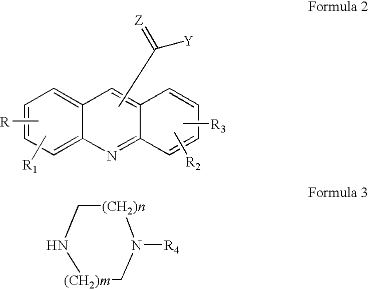
with an amine of the formula 3 in which R₄, m and n are as defined above, using, if appropriate, a condensing agent and/or a catalyst and diluents and auxiliaries, with formation of the desired acridine derivatives.
Synthesis of the Compounds According to the Invention

The compounds of the formula 1 can be obtained, for example, according to Schemes 1, 2 and 3 below:

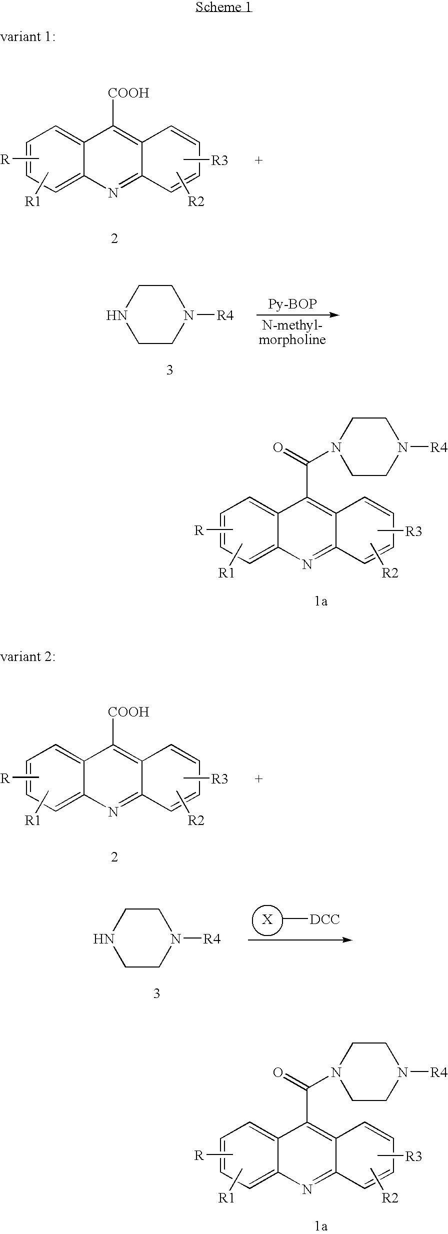

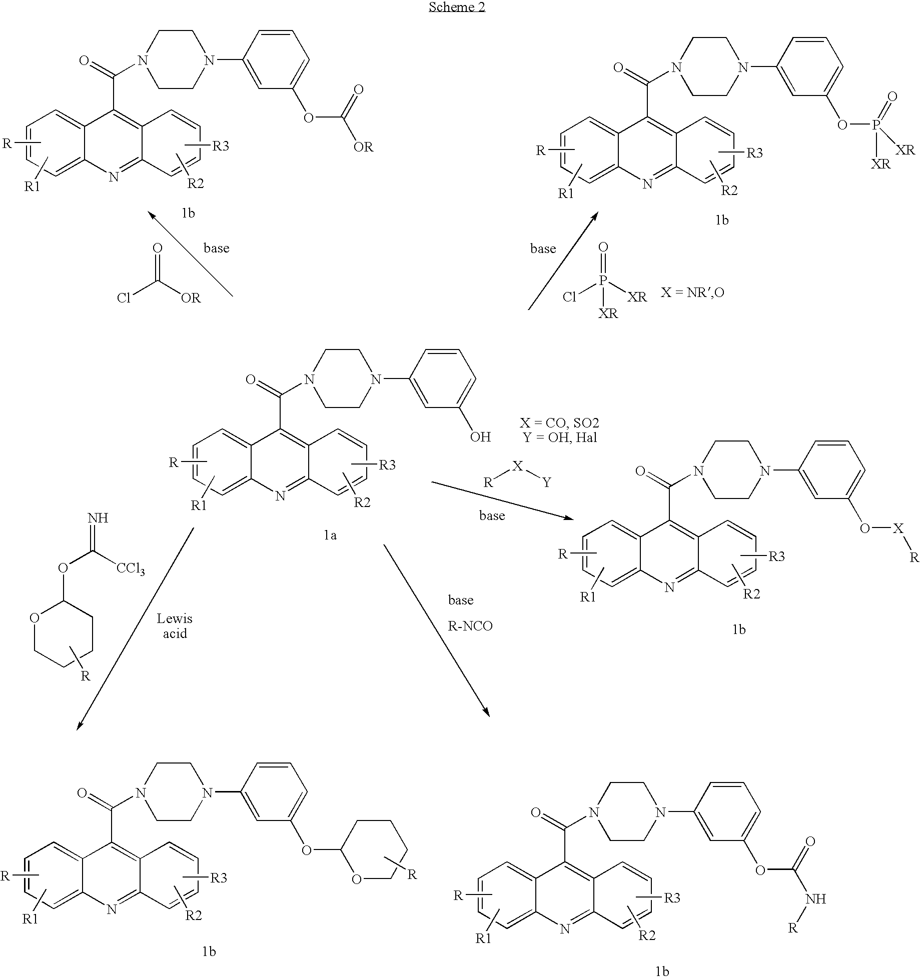

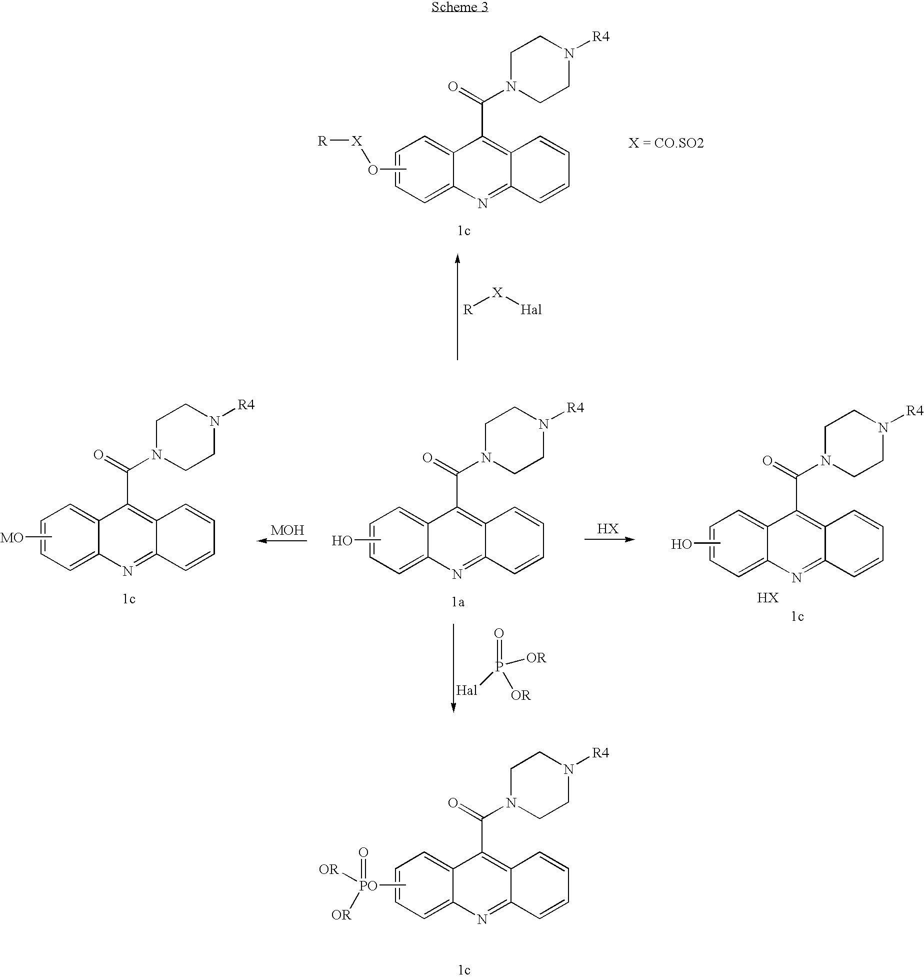

The starting compounds 2 and 3 are either commercially obtainable or can be prepared by procedures known per se. The starting materials 2 and 3 are valuable intermediate compounds for the preparation of the acridine derivatives of the formula 1 according to the invention.

The solvents and auxiliaries optionally to be used and reaction parameters such as reaction temperature and time to be used are known to the person skilled in the art on account of his/her expert knowledge.

The acridine derivatives of the formula 1 according to the invention are suitable as active compounds in medicaments, in particular as antitumor agents, for the treatment of humans and mammals. Mammals can be domestic animals such as horses, cows, dogs, cats; hares, sheep and the like.

The medicinal action of the acridine derivatives according to the invention can be based, for example on an interaction with the tubulin system by inhibition of tubulin polymerization. In addition, still further known and unknown mechanisms of action for the control of tumor cells are conceivable.

According to a further aspect of the invention, a process for the control of tumors in humans and in mammals is made available, which comprises administering at least one acridine derivative of the formula 1 to the human or a mammal in an amount effective for tumor treatment. The therapeutically effective dose of the respective acridine derivative according to the invention to be administered for the treatment depends, inter alia, on the nature and the stage of the oncosis, the age and sex of the patient, the manner of administration and the duration of treatment. The medicaments according to the invention can be administered as liquid, semisolid and solid pharmaceutical forms. This is carried out in the manner suitable in each case in the form of aerosols, powders and dusting powders, tablets, coated tablets, emulsions, foams, solutions, suspensions, gels, ointments, pastes, pills, pastilles, capsules or suppositories.

The pharmaceutical forms contain, in addition to at least one constituent according to the invention, depending on the pharmaceutical form employed, optionally excipients, such as, inter alia, solvents, solution accelerators, solubilizers, emulsifiers, wetting agents, antifoams, gel-forming agents, thickeners, film-forming agents, binders, buffers, salt-forming agents, drying agents, flow regulators, fillers, preservatives, antioxidants, colorants, mold release agents, lubricants, disintegrants, taste and odor corrigents. The selection of the excipients and the amounts thereof to be employed depends on the chosen pharmaceutical form and is orientated to the recipes known to the person skilled in the art.

The medicaments according to the invention can be administered in a suitable administration form to the skin, epicutaneously as a solution, suspension, emulsion, foam, ointment, paste or patch; via the oral and lingual mucosa, buccally, lingually or sublingually as a tablet, pastille, coated tablets, linctus or gargle; via the gastric and intestinal mucosa, enterally as a tablet, coated tablets, capsule, solution, suspension or emulsion; via the rectal mucosa, rectally as a suppository, rectal capsule or ointment; via the nasal mucosa, nasally as drops, ointments or spray; via the bronchial and alveolar epithelium, pulmonarily or by inhalation as an aerosol or inhalate; via the conjunctiva, conjunctivally as eyedrops, eye ointment, eye tablets, lamellae or eye lotion; via the mucosa of the genital organs, intravaginally as vaginal suppositories, ointments and flush, intrauterinely as a uterine pessary; via the efferent ureters, intraurethrally as a flush, ointment or medicated sound; into an artery, intraarterially as an injection; into a vein, intravenously as an injection or infusion, paravenously as an injection or infusion; into the skin, intracutaneously as an injection or implant; under the skin, subcutaneously as an injection or implant; into the muscle, intramuscularly as an injection or implant; into the abdominal cavity, intraperitoneally as an injection or infusion.

The compounds of the structure 1 according to the invention can be retarded in their pharmaceutical action with respect to practical therapeutic requirements by means of suitable measures. This aim can be achieved in a chemical and/or pharmaceutical way. Examples of the achievement of a prolongation of action are the use of implants, liposomes, sustained release forms, nanoparticle suspensions and “prodrugs” of the compounds according to the invention, the formation of poorly soluble salts and complexes or the use of crystal suspensions.

The compounds of the structure 1 according to the invention can be employed as an individual substance or in combination with further cytotoxic substances, such as, for example, cisplatin, carboplatin, doxorubicin, ifosfamide, cyclophosphamide, 5-FU, methotrexate or in combination with immunomodulators or antibodies and in particular in combination with inhibitors of signal transduction, such as, for example, herceptin, glivec or iressa.

Particularly preferred medicaments in this context are those which contain at least one compound from the following group of the aryl derivatives:
(1,3-dihydroxyacridin-9-yl )-[4-(6-methylpyridin-2-yl )piperazin-1-yl]methanone (1)
3-[4-(acridin-9-ylcarbonyl)piperazin-1-yl]phenyl isopropylcarbamate (2)
3-[4-(acridin-9-ylcarbonyl)piperazin-1-yl]phenyl acetate (3)
mono-{3-[4-(acridin-9-ylcarbonyl)piperazin-1-yl]phenyl}phosphate (4)
3-[4-(acridin-9-ylcarbonyl)piperazin-1-yl]phenyl methyl carbonate (5)
3-[4-(acridin-9-ylcarbonyl)piperazin-1-yl]phenyl 2-chloroethyl carbonate (6)
3-[4-(acridin-9-ylcarbonyl)piperazin-1-yl]phenyl (2-hydroxyethyl)carbamate (7)
[4-(3-chlorophenyl)piperazin-1-yl]-(1,3-dihydroxyacridin-9-yl)methanone (8)
[4-(6-chloropyridin-2-yl )piperazin-1-yl]-(1,3-dihydroxyacridin-9-yl)methanone (9)
(1,3-dihydroxyacridin-9-yl)-(2,3,5,6-tetrahydro-[1,2′]-bipyrazinyl-4-yl)methanone (10)
bis-{3-[4-(acridin-9-ylcarbonyl)piperazin-1-yl]phenyl}phosphate (11)
(1,3-dihydroxyacridin-9-yl)-[4-(6-methoxypyridin-2-yl)piperazin-1-yl]methanone (12)
(1,3-dihydroxyacridin-9-yl)-[4-(3-hydroxyphenyl)piperazin-1-yl]methanone (13)
(1,3-dihydroxyacridin-9-yl )-[4-(3-methoxyphenyl)piperazin-1-yl]methanone (14)
3-[4-(acridin-9-ylcarbonyl)piperazin-1-yl]phenyl methanesulfonate (15)
3-[4-(acridin-9-ylcarbonyl)piperazin-1-yl]phenyl 2,2-dimethyl-[1,3]-dioxolan-4-ylmethyl carbonate (16)
3-(diphenoxyphosphoryloxy)-9-[4-(3-methoxyphenyl)piperazin-1-yl-carbonyl]acridin-1-yl diphenyl phosphate (17)
3-acetoxy-9-[4-(3-methoxyphenyl)piperazin-1-ylcarbonyl]acridin-1-yl acetate (18)
bis-{3-[4-(acridin-9-ylcarbonyl)piperazin-1-yl]phenyl}carbonate (19)
3-[4-(acridin-9-ylcarbonyl)piperazin-1-yl]phenyl N,N-bis-(2-hydroxyethyl)succinate (20)
3-[4-(acridin-9-ylcarbonyl)piperazin-1-yl]phenyl 4-(4-methylpiperazin-1-yl)-4-oxobutyrate (21)
mono-{3-[4-(acridin-9-ylcarbonyl)piperazin-1-yl]phenyl}glutarate (22)
3-[4-(acridin-9-ylcarbonyl)piperazin-1-yl]phenyl 5-(2,2-dimethyl-[1,3]dioxolan-4-yl)-2,2-dimethyltetrahydrofuro[2,3-d][1,3]dioxol-6-yl succinate (23)
3-[4-(acridin-9-ylcarbonyl)piperazin-1-yl]phenyl 5-[5-(2,2-dimethyl-[1,3]-dioxolan-4-yl)-2,2-dimethyltetrahydrofuro[2,3-d][1,3]dioxol-6-yloxy]-pentanoate (24)
3-[4-(acridin-9-ylcarbonyl)piperazine-1-yl]phenyl 2,3,5-trihydroxy-6-hydroxymethyltetrahydropyran-4-yl succinate (25)
3-[4-(acridin-9-ylcarbonyl)piperazin-1-yl]phenyl 5-(2,3,5-trihydroxy-6-hydroxymethyltetrahydropyran-4-yloxy)pentanoate (26)
3-[4-(acridin-9-ylcarbonyl)piperazin-1-yl]phenyl [1,4′]bispiperidinyl-1′-carboxylate (27)
3-[4-(acridin-9-ylcarbonyl)piperazin-1-yl]phenyl 4-morpholin-4-yl-piperidine-1-carboxylate (28)
3-[4-(acridin-9-ylcarbonyl)piperazin-1-yl]phenyl (2-morpholin-4-ylethyl)carbamate (29)
3-[4-(acridin-9-ylcarbonyl)piperazin-1-yl]phenyl (2-chloroethyl)carbamate (30)
3-[4-(acridin-9-ylcarbonyl)piperazin-1-yl]phenyl diethylcarbamate (31)
bis(dimethylamide) mono-{3-[4-acridin-9-ylcarbonyl)piperazin-1-yl]phenyl}phosphate (32)
mono-{3-[4-(acridin-9-ylcarbonyl)piperazin-1-yl]phenyl}phosphate disodium salt (33)
3-[4-acridin-9-ylcarbonyl)piperazin-1-yl]phenyl nonadecanoate (34)
1-hydroxy-9-[4-(3-methoxyphenyl)piperazin-1-ylcarbonyl]acridin-3-yl diphenyl phosphate (35)
3-hydroxy-9-[4-(3-methoxyphenyl)piperazin-1-ylcarbonyl]acridin-1-yl diphenyl phosphate (36)
(2-hydroxyacridin-9-yl)-[4-(3-methoxyphenyl)piperazin-1-yl]methanone (37)
(2-hydroxyacridin-9-yl)-[4-(3-hydroxyphenyl)piperazin-1-yl]methanone (38)
(1,3-dihydroxyacridin-9-yl)-[4-(4-methylpyridin-2-yl)piperazin-1-yl]methanone (39)
3-acetoxy-9-[4-(6-methylpyridin-2-yl)piperazin-1-ylcarbonyl]acridin-1-yl acetate (40)
3-acetoxy-9-[4-(6-methoxypyridin-2-yl)piperazin-1-ylcarbonyl]acridin-1-yl acetate (41)
   and can be present both as a free base and as salts of physiologically tolerable acids.

According to this general procedure for steps 1, 2 and 3, on which synthesis schemes 1, 2 and 3 are based, the following compounds were synthesized which follow from the list below with statement of the respective chemical name. The analytical characterization of the compounds according to the invention was carried out by means of their melting points or by ¹H- and ³¹P-NMR spectroscopy and/or mass spectrometry.

The chemicals and solvents employed were obtained commercially from the conventional suppliers (Acros, Avocado, Aldrich, Fluka, Lancaster, Maybridge, Merck, Sigma, TCI etc.) or synthesized.

The invention is intended to be illustrated in greater detail with the aid of the following examples, without being restricted thereto.

### EXAMPLE 1

### Reaction as in Scheme 1, Variant 1

### Acridin-9-yl-[4-(3-hydroxyphenyl)piperazin-1-yl]methanone

2.17 g (21.5 mMol) of N-methylmorpholine, 2.4 g (13.44 mMol) of N-(3-hydroxyphenyl)piperazine and 7.69 g (14.78 mMol) of Py-BOP (1-benzotriazolyltripyrrolidinophosphonium hexafluorophosphate) were added successively to a solution of 3 g (13.44 mMol) of acridine-9-carboxylic acid hydrate in 30 ml of dimethylformamide. The mixture was stirred at room temperature for 4 hours and allowed to stand at room temperature overnight, the dimethylformamide was distilled off under reduced pressure and the residue was purified on a silica gel column (silica gel 60, Merck AG, Darmstadt, Germany) using the mobile phase dichloromethane/methanol (95:5 v/v).

Yield: 3.01 g (57.8% of theory)

m.p.: 143° C.

### EXAMPLE 2

### Reaction as in Scheme 1, Variant 2

### (1,3-Dihydroxyacridin-9-yl)-[4-(6-methylpyridin-2-yl)piperazin-1-yl]-methanone (1)

6.66 g (11.06 mMol) of polymer-bound N-benzyl-N-cyclohexylcarbodiimide (1.66 mMol/g) were added to a solution of 1.8 g (7.05 mMol) of 1,3-dihydroxyacridine-9-carboxylic acid in 40 ml of dimethylformamide and the mixture was heated at 60° C. and allowed to react for 30 minutes. 1.03 g (5.64 mMol) of 1-(2-(6-methylpyridinyl))piperazine were added, and the mixture was allowed to react for a further 4 hours. The mixture was then allowed to cool, the resin was removed, the dimethylformamide was distilled off under reduced pressure and the residue was purified on a silica gel column (silica gel 60, Merck AG, Darmstadt, Germany) using the mobile phase dichloromethane/methanol (95:5 v/v).

Yield: 2.3 g (74.8% of theory)

¹H-NMR (DMSO-d6) δ=10.9 (s, 1H), 10.3 (s, 1H), 7.97 (d, 1H), 7.84 (d, 1H), 7.75 (t, 1 H), 7.5-7.4 (m, 2H), 6.86 (d, 1H), 6.61 (d, 1H), 6.56 (d, 1H), 4.05 (m, 1H), 3.8 (m, 1H), 3.7 (m, 1H), 3.6 (m, 1H), 3.5 (m, 1H), 3.15 (m, 1H), 3.05 (m, 2H), 2.25 (s, 3H) ppm.

### EXAMPLE 3

### Reaction as in Scheme 2

### 3-[4-(Acridin-9-ylcarbonyl)piperazin-1-yl]phenyl isopropylcarbamate (2)

40 μl (0.39 mMol) of triethylamine and 24 μl (0.29 mMol) of isopropyl isocyanate were added successively to a suspension of 100 mg (0.26 mMol) of acridin-9-yl-[4-(3-hydroxyphenyl)piperazin-1-yl]methanone in 15 ml of dichloromethane. After 18 h of stirring at room temperature, the solvent was removed under reduced pressure and the residue was purified on a silica gel column using the mobile phase dichloromethane/methanol (99:1 v/v).

Yield: 95 mg (78% of theory).

m.p.: 197-198° C.

¹H-NMR (DMSO-d₆) δ=8.24 (d, 2H), 7.99 (d, 2H), 7.91 (dd, 2H), 7.71 (dd, 2H), 7.59 (d, 1H), 7.18 (dd, 1H), 6.75 (dd, 1H), 6.62 (s, 1H), 6.52 (d, 1H), 4.10-4.12 (m, 2H), 3.59-3.65 (m, 1H), 3.46-3.48 (m, 2H), 3.10-3.12 (m, 2H), 2.95-2.97 (m, 2H), 1.10 (d, 6 H) ppm.

### EXAMPLE 4

### Reaction as in Scheme 2

### 3-[4-(Acridin-9-ylcarbonyl)piperazin-1-yl]phenyl acetate (3)

40 μl (0.39 mMol) of triethylamine and 27 μl (29 mMol) of acetic anhydride were added successively to a suspension of 100 mg (0.26 mMol) of acridin-9-yl-[4-(3-hydroxyphenyl)piperazin-1-yl]methanone in 15 ml of dichloromethane. After 18 h of stirring at room temperature, the solvent was removed under reduced pressure and the residue was purified on a silica gel column using the mobile phase dichloromethane/methanol (99:1 v/v).

Yield: 81 mg (73% of theory).

m.p.: 162° C.

¹H-NMR (DMSO-d₆) δ=8.24 (d, 2H), 7.99 (d, 2H), 7.92 (dd, 2H), 7.71 (dd, 2H), 7.22 (dd, 1H), 6.81 (dd, 1H), 6.67 (s, 1H), 6.54 (dd, 1H), 4.10-4.12 (m, 2H), 3.47-3.50 (m, 2H), 3.10-3.13 (m, 2H), 2.95-2.98 (m, 2H), 2.22 (s, 3 H) ppm.

### EXAMPLE 5

### Reaction as in Scheme 2

### Bis(dimethylamide) mono-{3-[4-(acridin-9-ylcarbonyl)piperazin-1-yl]phenyl}phosphate (32):

A solution of 364 mg (0.94 mMol) of acridin-9-yl-[4-(3-hydroxyphenyl)piperazin-1-yl]methanone in 10 ml of pyridine was reacted with 360 mg (1.90 mMol) of bis(dimethylamide) phosphoryl chloride, 214 mg (1.4 mMol) of DBU and 232 mg (1.90 mMol) of DMAP at room temperature for 2 hours. The reaction solution was then concentrated under reduced pressure, giving, as residue, a brown oil. The crude product was dissolved in a little DMF and purified by two column chromatographies (Geduran Si 60, column L 280, (/) 25, mobile phase dichloromethane/methanol 95:5). This gave 433 mg of bis(dimethylamide) mono-{3-[4-(acridin-9-ylcarbonyl)piperazin-1-yl]phenyl}phosphate in a purity of 88%.

¹H-NMR (DMSO-d₆): d=8.25 (d, 2H), 8.02 (d, 2H), 7.96 (t, 2H), 7.74 (t, 2H), 7.17 (t, 1H), 6.72 (d, 1H), 6.67 (s, 1H), 6.60 (d, 1H), 4.12 (m, 2H), 3.47 (m, 2H), 3.14 (m, 2H), 2.95 (m, 2H), 2.60 (s, 6H), 2.58 (s, 6H) ppm.

### EXAMPLE 6

### Reaction as in Scheme 2

### Mono-{3-[4-(acridin-9-ylcarbonyl)piperazin-1-yl]phenyl}phosphate (4)

370 mg of the bis(dimethylamide) mono-{3-[4-(acridin-9-ylcarbonyl)piperazin-1-yl]phenyl}phosphate were then taken up in 1 ml of water and 9 ml of TFA and stirred at room temperature for 2 h.

The reaction solution was then concentrated under reduced pressure and lyophilized. The residue was purified by preparative high pressure chromatography (HPLC—JO, RP18, 250-50, 12 μm, flow rate 60 ml/min; 10% B—100% B over 30 min), giving 315 mg of mono-{3-[4-(acridin-9-ylcarbonyl)piperazin-1-yl]phenyl}phosphate.

Yield: 315 mg (84.7% of theory)

M: 463 g/mol; molar mass found 464.1 [M+H]⁺

¹H-NMR (DMSO-d6): δ=8.24 (d, 2H), 8.00 (d, 2H), 7.93 (t, 2H), 7.72 (t, 2H), 7.16 (t, 1H), 6.71 (d, 1H), 6.68 (s, 1H), 6.63 (d, 1H), 4.13-4.11(m, 2H), 3.47-3.45 (m, 2H), 3.14-3.12 (m, 2H), 2.95-2.93 (m, 2H).

³¹P-NMR (DMSO-d6): δ=−6.44 ppm

### EXAMPLE 7

### Reaction as in Scheme 2

### Mono-{3-[4-(acridin-9-ylcarbonyl)piperazin-1-yl]phenyl}phosphate disodium salt (33)

4060 mg (9.00 mMol) of mono-{3-[4-(acridin-9-ylcarbonyl)piperazin-1-yl)phenyl) phosphate were suspended in 200 ml of water, 700 mg (18 mMol) of an aqueous sodium hydroxide solution (1 M solution) were added and the mixture was stirred at room temperature for 45 minutes, resulting in the formation of a clear yellow solution. The reaction solution was then filtered off with suction through a membrane filter, and the aqueous solution was lyophilized. The residue was then dried over calcium chloride under reduced pressure. The residue obtained were 4160 mg of mono-{3-[4-(acridin-9-ylcarbonyl)piperazin-1-yl]phenyl}phosphate disodium salt.

Yield: 4160 mg (93.6% of theory)

M: 463 g/mol; molar mass found 464.1 [M+H]⁺

### EXAMPLE 8

### Reaction as in Scheme 2

### 3-[4-(Acridin-9-ylcarbonyl)piperazin-1-yl]phenyl methyl carbonate (5)

40 μl (0.39 mMol) of triethylamine and 22 μl (0.29 mMol) of methyl chloroformate were added successively to a suspension of 100 mg (0.26 mMol) of acridin-9-yl-[4-(3-hydroxyphenyl)piperazin-1-yl]methanone in 15 ml of dichloromethane. After 18 h of stirring at room temperature, the solvent was removed under reduced pressure and the residue was purified on a silica gel column using the mobile phase dichloromethane/methanol (99:1 v/v).

Yield: 99 mg (86.0% of theory).

m.p.: 183-184° C.

¹H-NMR (DMSO-d₆) δ=8.24 (d, 2H), 7.99 (d, 2H), 7.92 (dd, 2H), 7.71 (dd, 2H), 7.24 (dd, 1H), 6.84 (dd. 1H), 6.78 (s, 1H), 6.64 (dd, 1H), 4.10-4.12 (m, 2H), 3.80 (s, 3H), 3.49-3.52 (m, 2H), 3.10-3.13 (m, 2H), 2.97-3.00 (m, 2H) ppm.

### EXAMPLE 9

### Reaction as in Scheme 2

### 3-[4-(Acridin-9-ylcarbonyl)piperazin-1-yl]phenyl methanesulfonate (15)

A solution of 0.15 g (0.39 mMol) of acridin-9-yl-[4-(3-hydroxyphenyl)piperazin-1-yl]methanone in 15 ml of pyridine was reacted at room temperature with 0.07 g (0.59 mMol) of methanesulfonyl chloride for 4 hours. The pyridine was then distilled off under reduced pressure and the residue was purified on a silica gel column (silica gel 60, Merck AG, Darmstadt, Germany) using the mobile phase dichloromethane/methanol (95:5 v/v).

Yield: 8 mg (4.1% of theory)

¹H-NMR (DMSO-d6) δ=8.25 (d, 2H), 7.9-8.05 (m, 5H), 7.7 (t, 2H), 7.3 (t, 1H), 6.95 (d, 1H), 6.75 (d, 1H), 4.13 (m, 2H), 3.55 (m, 2H), 3.33 (s, 3H), 3.12 (m, 2H), 3.05 (m, 2H) ppm.

### EXAMPLE 10

### Reaction as in Scheme 3

### 3-(Diphenoxyphosphoryloxy)-9-[4-(3-methoxyphenyl)piperazin-1-yl-carbonyl]acridin-1-yl diphenyl phosphate (17)

A solution of 0.46 g (1.1 mMol) of (1,3-dihydroxyacridin-9-yl)-[4-(3-methoxyphenyl)piperazin-1-yl]methanone in 30 ml of pyridine was cooled to T=−10° C. and reacted at this temperature with 0.72 g (2.68 mMol) of diphenyl chlorophosphate and 0.58 g (4.5 mMol) of diisopropylethylamine for 1.5 hours. The mixture was then diluted with 10 ml of dichloromethane and extracted three times with in each case 10 ml of water. The solvent of the organic phase was distilled off under reduced pressure and the residue was purified on a silica gel column (silica gel 60, Merck AG, Darmstadt, Germany) using the mobile phase dichloromethane/methanol (97:3 v/v).

Yield: 134 mg (13.3% of theory)

¹H-NMR (DMSO-d6) δ=8.25 (d, 1H), 8.06-8.0 (m, 3H), 7.78 (m, 2H), 7.46-7.23 (m, 22 H), 7.1 (t, 1H), 6.42 (m, 1H), 3.7-3.1 (m, 11H) ppm.

³¹P-NMR (DMSO-d6): δ=−18.2, −17.8 ppm

### EXAMPLE 11

### Reaction as in Scheme 3

### 3-Acetoxy-9-[4-(3-methoxyphenyl)piperazin-1-ylcarbonyl]acridin-1-yl acetate (18)

A solution of 0.30 g (0.7 mMol) of (1,3-dihydroxyacridin-9-yl)-[4-(3-methoxyphenyl)piperazin-1-yl]methanone in 20 ml of dimethylformamide was reacted with 0.18 g (1.75 mMol) of triethylamine for 30 minutes and then with 0.11 g (1.4 mMol) of acetic anhydride for 0.75 hours. The mixture was then diluted with 10 ml of dichloromethane and extracted three times with in each case 10 ml of water. The solvent of the organic phase was distilled off under reduced pressure and the residue was purified on a silica gel column (silica gel 60, Merck AG, Darmstadt, Germany) using the mobile phase dichloromethane/methanol (97:3 v/v).

Yield: 308 mg (81.5% of theory)

¹H-NMR (DMSO-d6) δ=8.24 (d, 1H), 8.0-7.9 (m, 3H), 7.72 (t, 1H), 7.12 (t, 1H), 6.53-6.38 (m, 4H), 4.35 (m, 1-H), 3.84 (m,1 H), 3.73 (m, 4H), 3.56 (m, 1H), 3.08-2.96 (m, 3H), 2.74 (m, 1H), 2.38, 2.36 (2s, 6H) ppm.

The following compounds of the formula 1 were synthesized analogously to the synthesis route in scheme 1, 2 and 3:

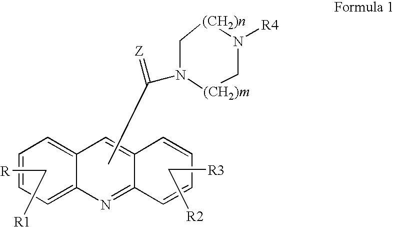

### EXAMPLE 12

### 3-[4-(Acridin-9-ylcarbonyl)piperazin-1-yl]phenyl 2-chloroethyl carbonate (6)

m.p.: 162-163° C.

¹H-NMR (DMSO-d₆) δ=8.24 (d, 2H), 7.99 (d, 2H), 7.92 (dd, 2H), 7.71 (dd, 2H), 7.25 (dd, 1H), 6.85 (dd, 1H), 6.81 (s, 1H), 6.66 (dd, 1H), 4.42-4.45 (m, 2H), 4.10-4.12 (m, 2H), 3.89-3.92 (m, 2H), 3.49-3.53 (m, 2H), 3.10-3.13 (m, 2H), 2.98-3.00 (m, 2H) ppm.

### EXAMPLE 13

### 3-[4-(Acridin-9-ylcarbonyl)piperazin-1-yl]phenyl (2-hydroxyethyl)carbamate (7)

m.p.: decomposition from 116° C.

¹H-NMR (DMSO-d₆) δ=8.24 (d, 2H), 7.99 (d, 2H), 7.92 (dd, 2H), 7.71 (dd, 2H), 7.61 (t, 1H),7.18 (dd, 1H), 6.76 (dd, 1H), 6.62 (s, 1H), 6.52 (dd, 1H), 4.69 (t, 1H), 4.10-4.12 (m, 2H), 3.41-3.49 (m, 4H), 3.07-3.13 (m, 4H), 2.94-2.97 (m, 2H) ppm.

### EXAMPLE 14

### [4-(3-Chlorophenyl)piperazin-1-yl]-(1,3-dihydroxyacridin-9-yl)methanone (8)

¹H-NMR (DMSO-d₆) δ=10.95 (s, 1H), 10.3 (s, 1H), 7.94 (d, 1H), 7.83 (d, 1H), 7.76 (t, 1H), 7.48 (t, 1H), 7.23 (t, 1H), 6.96 (m, 1H), 6.9 (m, 1H), 6.86 (s, 1H), 6.82 (m, 1H), 6.55 (s, 1H), 4.1 (m, 1H), 3.77 (m, 1H), 3.5 (m, 1H), 3.22 (m, 1H), 3.07 (m, 2H), 3.15 (m, 1H), 2.86 (m, 1H) ppm.

### EXAMPLE 15

### [4-(6-Chloropyridin-2-yl)piperazin-1-yl]-(1,3-dihydroxyacridin-9-yl)-methanone (9)

¹H-NMR (DMSO-d₆) δ=10.9 (s, 1H), 10.3 (s, 1H), 7.96 (d, 1H), 7.83 (d, 1H), (t, 1H), 7.57 (t, 1H), 7.47 (t, 1H), 6.87 (m, 1 H), 6.79 (d, 1H), 6.7 (d, 1H), 4.06 (m, 1H), 3.84 (m, 1H), 3.65-3.78 (m, 2H), 3.53 (m, 1H), 3.2 (m, 1H), 3.05 (m, 2H) ppm.

### EXAMPLE 16

### (1,3-Dihydroxyacridin-9-yl)-(2,3,5,6-tetrahydro-[1,2′]-bipyrazinyl-4-yl)-methanone (10)

¹H-NMR (DMSO-d₆) δ=10.95 (s, 1H), 10.3 (s, 1H), 8.3 (s, 1H), 8.1 (s, 1H), 7.96 (d, 1H), 7.87 (d, 1H), 7.83 (d, 1H) 7.75 (t, 1H), 7.47 (t, 1H), 6.87 (m, 1H), 6.53 (m, 1H), 4.05 (m, 1H), 3.85 (m, 1H), 3.73 (m, 2H), 3.62 (m, 1H), 3.1 (m, 2H) ppm.

### EXAMPLE 17

### bis-{3-[4-(Acridin-9-ylcarbonyl)piperazin-1-yl]phenyl}phosphate (11)

ESIMS: 829.2 [M+H]⁺ ¹H-NMR (DMSO-d₆): δ=8.26 (d, 4H), 8.00 (d, 4H), 7.98 (t, 4H), 7.74 (t, 4H), 7.13 (t, 2H), 6.67-6.62 (m, 6H), 4.07-4.05(m, 4H), 3.41-3.39 (m, 4H), 3.09-3.07 (m, 4H), 2.87-2.85 (m, 4H).

³¹P-NMR (DMSO-d6): δ=−12.18 ppm

### EXAMPLE 18

### (1,3-Dihydroxyacridin-9-yl)-[4-(6-methoxypyridin-2-yl)piperazin-1-yl]-methanone (12)

¹H-NMR (DMSO-d6) δ=10.9 (s, 1H), 10.3 (s, 1H), 7.96 (d, 1H), 7.82 (d, 1H), 7.75 (t, 1H), 7.45 (m, 2H), 6.86 (s, 1H), 6.33 (d, 1H), 6.07 (d, 1H), 4.05 (m, 1H), 3.84 (m, 1H), 3.73 (m, 4H), 3.64 (m,1H), 3.54 (m, 1H), 3.05 (m, 2H) ppm.

### EXAMPLE 19

### (1,3-Dihydroxyacridin-9-yl)-[4-(3-hydroxyphenyl)piperazin-1-yl]methanone (13)

¹H-NMR (DMSO-d6) δ=10.9 (s, 1H), 10.3 (s, 1H), 9.16 (s, 1H), 7.96 (m, 1H), 7.8 (t,1H), 7.75 (t, 1H), 7.46 (t, 1H), 6.96 (t, 1H), 6.85 (m, 1H), 6.56 (m, 1H), 6.36 (m, 1H), 6.3 (m, 1H), 6.23 (m, 1H), 4.1 (m, 1H), 3.75 (m, 1H), 3.42 (m, 1H), 3.22 (m, 1H), 2.9 (m, 1H), 2.76 (m, 1H), 2.27 (m, 1H) ppm.

### EXAMPLE 20

### (1,3-Dihydroxyacridin-9-yl)-[4-(3-methoxyphenyl)piperazin-1-yl]methanone (14)

¹H-NMR (DMSO-d6) δ=10.95 (s, 1H), 10.3 (s, 1H), 7.97 (m, 2H), 7.82 (m, 1H), 7.75 (t, 1H), 7.47 (t, 1H), 7.1 (t, 1H), 6.53 (m, 2H), 6.45 (m, 1H), 6.38 (m, 1H), 4.1 (m, 1H), 3.68-3.78 (m, 5H), 3.47 (m, 1H), 3.15 (m, 1H), 3.05 (m, 1H), 2.84 (m, 1H) ppm.

### EXAMPLE 21

### 3-[4-(Acridin-9-ylcarbonyl)piperazin-1-yl]phenyl 2,2-dimethyl-[1,3]-dioxolan-4-ylmethyl carbonate (16)

m.p.: 107-108° C.

¹H-NMR (DMSO-d₆) δ=8.24 (d, 2H), 7.99 (d, 2H), 7.92 (dd, 2H), 7.71 (dd, 2H), 7.24 (dd, 1H), 6.84 (dd, 1H), 6.79 (s, 1H), 6.55 (dd, 1H), 4.31-4.36 (m, 1H), 4.27 (dd, 1H), 4.14 (dd, 1H), 4.10-4.12 (m, 2H), 3.73 (dd, 1H), 3.10-3.13 (m, 2H), 2.97-2.30 (m, 2H), 1.34 (s, 3H), 1.28 (s, 3H) ppm.

### EXAMPLE 22

### bis-{3-[4-(Acridin-9-ylcarbonyl)piperazin-1-yl]phenyl}carbonate (19)

ESIMS: 793.2 [M+H]⁺, 397.2 [M+2H]⁺⁺.

¹H-NMR (DMSO-d₆) δ=8.24 (d, 4H), 7.99 (d, 4H), 7.92 (dd, 4H), 7.70 (dd, 4H), 7.27 (dd, 2H), 6.90 (s, 2H), 6.87 (dd, 2H), 6.74 (dd, 2H), 4.10-4.13 (m, 4H), 3.50-3.53 (m, 4H), 3.11-3.13 (m, 4H), 2.98-3.01 (m, 4H) ppm.

### EXAMPLE 23

### 3-[4-(Acridin-9-ylcarbonyl)piperazin-1-yl]phenyl N,N-bis-(2-hydroxy-ethyl)succinate (20)

¹H-NMR (DMSO-d₆) δ=8.24 (d, 2H), 7.99 (d, 2H), 7.92 (dd, 2H), 7.71 (dd, 2H), 7.22 (dd, 1H), 6.81 (dd, 1H), 6.63 (s, 1H), 6.52 (dd, 1H), 4.85 (t, 1H), 4.66 (t, 1H), 4.10-4.12 (m, 2H), 3.40-3.55 (m, 8H), 3.32-3.34 (m, 2H), 3.10-3.13 (m, 2H), 2.94-2.97 (m, 2H), 2.67-2.75 (m, 4H) ppm.

### EXAMPLE 24

### 3-[4-(Acridin-9-ylcarbonyl)piperazin-1-yl]phenyl 4-(4-methylpiperazin-1-yl)-4-oxobutyrate (21)

H-NMR (DMSO-d₆) δ=8.24 (d, 2H), 7.99 (d, 2H), 7.92 (dd, 2H), 7.71 (dd, 2H), 7.22 (dd, 1H), 6.81 (dd, 1H), 6.62 (s, 1H), 6.52 (dd, 1H), 4.10-4.12 (m, 2H), 3.41-3.49 (m, 6H), 3.10-3.13 (m, 2H), 2.94-2.97 (m, 2H), 2.64-2.73 (m, 4H), 2.14-2.38 (m, 7H) ppm.

### EXAMPLE 25

### Mono-{3-[4-(acridin-9-ylcarbonyl)piperazin-1-yl]phenyl}glutarate (22)

¹H-NMR (DMSO-d₆) δ=12.14 (s, 1H), 8.24 (d, 2H), 7.99 (d, 2H), 7.92 (dd, 2H), 7.71 (dd, 2H), 7.22 (dd, 1H), 6.81 (dd, 1H), 6.68 (s, 1H), 6.53 (dd, 1H), 4.09-4.12 (m, 2H), 3.47-3.50 (m, 2H), 3.10-3.13 (m, 2H), 2.95-2.99 (m, 2H), 2.57 (t, 2H), 2.32 (t, 2H), 1.80-1.84 (m, 2H) ppm.

### EXAMPLE 26

### 3-[4-(Acridin-9-ylcarbonyl)piperazin-1-y;]phenyl 5-(2,2-dimethyl-[1,3]dioxolan-4-yl)-2,2-dimethyltetrahydrofuro[2,3-d][1,3]dioxol-6-yl succinate (23)

H-NMR (DMSO-d₆) δ=8.24 (d, 2H), 7.99 (d, 2H), 7.92 (dd, 2H), 7.71 (dd, 2H), 7.23 (dd, 1H), 6.83 (dd, 1H), 6.65 (s, 1H), 6.54 (dd, 1H), 5.86 (d, 1H), 5.05 (d, 1H), 4.49 (d, 1H), 4.14-4.22 (m, 2H), 4.09-4.13 (m, 2H), 4.00 (dd, 1H), 3.85 (dd, 1H), 3.46-3.49 (m, 2H), 3.10-3.13 (m, 2H), 2.95-2.98 (m, 2H), 2.80-2.84 (m, 2H), 2.68-2.71 (m, 2H), 1.40 (s, 3H), 1.29 (s, 3H), 1.20 (s, 3H), 1.19 (s, 3H) ppm.

### EXAMPLE 27

### 3-[4-(Acridin-9-ylcarbonyl)piperazin-1-yl]phenyl 5-[5-(2,2-dimethyl-[1,3]-dioxolan-4-yl)-2,2-dimethyltetrahydrofuro[2,3-d][1,3]dioxol-6-yloxy]-pentanoate (24)

¹H-NMR (DMSO-d₆) δ=8.24 (d, 2H), 7.99 (d, 2H), 7.92 (dd, 2H), 7.71 (dd, 2H), 7.22 (dd, 1H), 6.81 (dd, 1H), 6.64 (s, 1H), 6.52 (dd, 1H), 5.82 (d, 1H), 4.61 (d, 1H), 4.20-4.24 (m, 1H), 4.10-4.12 (m, 2H), 4.01-4.04 (m, 1H), 3.94-3.97 (m, 1H), 3.76-3.80 (m, 2H), 3.39-3.66 (m, 4H), 3.10-3.13 (m, 2H), 2.95-2.98 (m, 2H), 2.54-2.57 (m, 2H), 1.53-1.69 (m, 4H), 1.38 (s, 3H), 1.30 (s, 3H), 1.24 (s, 3H), 1.23 (s, 3H) ppm.

### EXAMPLE 28

### 3-[4-(Acridin-9-ylcarbonyl)piperazin-1-yl]phenyl 2,3,5-trihydroxy-6-hydroxymethyltetrahydropyran-4-yl succinate (25)

¹H-NMR (MeOH-d₄) δ=8.30 (d, 2H), 8.13 (d, 2H), 8.05 (dd, 2H), 7.82 (dd, 2H), 7.25 (dd, 1H), 6.86 (dd, 1H), 6.73 (s, 1H), 6.62 (dd, 1H), 4.54-5.28 (m, 2H), 4.25-4.29 (m, 2H), 3.64-3.87 (m, 3H), 3.43-3.55 (m, 4H), 3.25-3.28 (m, 2H), 3.02-3.05 (m, 2H), 2.82-2.90 (m, 4H) ppm.

### EXAMPLE 29

### 3-[4-(Acridin-9-ylcarbonyl)piperazin-1-yl]phenyl 5-(2,3,5-trihydroxy-6-hydroxymethyltetrahydropyran-4-yloxy)pentanoate (26)

¹H-NMR (MeOH-d₄) δ=8.32 (d, 2H), 8.16 (d, 2H), 8.10 (dd, 2H), 7.85 (dd, 2H), 7.25 (dd, 1H), 6.86 (dd, 1H), 6.69 (s, 1H), 6.60 (dd, 1H), 5.08 (d, 0.5H), 4.47 (d, 0.5H), 4.26-4.29 (m, 2H), 3.15-3.90 (m, 12H), 3.02-3.05 (m, 2H), 2.60-2.63 (m, 2H), 1.70-1.86 (m, 4H) ppm.

### EXAMPLE 30

### 3-[4-(Acridin-9-ylcarbonyl)piperazin-1-yl]phenyl[1,4′]bispiperidinyl-1′-carboxylate (27)

¹H-NMR (DMSO-d₆) δ=8.24 (d, 2H), 7.99 (d, 2H), 7.92 (dd, 2H), 7.71 (dd, 2H), 7.19 (dd, 1H), 6.77 (dd, 1H), 6.65 (s, 1H), 6.54 (dd, 1H), 3.97-4.17 (m, 4H), 3.46-3.49 (m, 2H), 3.09-3.13 (m, 2H), 2.75-2.99 (m, 4H), 2.40-2.49 (m, 5H), 1.71-1.77 (m, 2H), 1.34-1.51 (m, 8H) ppm.

### EXAMPLE 31

### 3-[4-(Acridin-9-ylcarbonyl)piperazin-1-yl]phenyl 4-morpholin-4-yl-piperidine-1-carboxylate (28)

¹H-NMR (DMSO-d₆) δ=8.24 (d, 2H), 7.99 (d, 2H), 7.92 (dd, 2H), 7.71 (dd, 2H), 7.19 (dd, 1H), 6.77 (dd, 1H), 6.65 (s, 1H), 6.54 (dd, 1H), 3.95-4.13 (m, 4H), 3.55-3.59 (m, 4H), 3.46-3.49 (m, 2H), 3.09-3.13 (m, 2H), 2.81-3.03 (m, 4H), 2.43-2.49 (m, 4H), 2.33-2.39 (m, 1H), 1.78-1.83 (m, 2H), 1.30-1.43 (m, 2H) ppm.

### EXAMPLE 32

### 3-[4-(Acridin-9-ylcarbonyl)piperazin-1-yl]phenyl (2-morpholin-4-yl-ethyl)carbamate (29)

¹H-NMR (DMSO-d₆) δ=8.24 (d, 2H), 7.99 (d, 2H), 7.92 (dd, 2H), 7.71 (dd, 2H), 7.58 (t, 1H), 7.19 (dd, 1H), 6.76 (dd, 1H), 6.62 (s, 1H), 6.52 (dd, 1H), 4.19-4.12 (m, 2H), 3.54-3.57 (m, 4H), 3.46-3.49 (m, 2H), 3.10-3.17 (m, 4H), 2.94-2.97 (m, 2H), 2.35-2.41 (m, 6H) ppm.

### EXAMPLE 33

### 3-[4-(Acridin-9-ylcarbonyl)piperazin-1-yl]phenyl (2-chloroethyl)carbamate (30)

¹H-NMR (DMSO-d₆) δ=8.24 (d, 2H), 7.99 (d, 2H), 7.97 (t, 1H), 7.92 (dd, 2H), 7.71 (dd, 2H), 7.19 (dd, 1H), 6.77 (dd, 1H), 6.64 (s, 1H), 6.54 (dd, 1H), 4.09-4.12 (m, 2H), 3.65 (t, 2H), 3.46-3.49 (m, 2H), 3.35-3.68 (m, 2H), 3.10-3.13 (m, 2H), 2.95-2.98 (m, 2H) ppm.

### EXAMPLE 34

### 3-[4-(Acridin-9-ylcarbonyl)piperazin-1-yl]phenyl diethylcarbamate (31)

¹H-NMR (DMSO-d₆) δ=8.24 (d, 2H), 7.99 (d, 2H), 7.92 (dd, 2H), 7.71 (dd, 2H), 7.19 (dd, 1H), 6.77 (dd, 1H), 6.65 (s, 1H), 6.54 (dd, 1H), 4.09-4.12 (m, 2H), 3.47-3.50 (m, 2H), 3.37 (q, 2H), 3.27 (q, 2H), 3.10-3.13 (m, 2H), 2.95-2.98 (m, 2H), 1.17 (t, 3H), 1.09 (t, 3H) ppm.

### EXAMPLE 35

### 3-[4-Acridin-9-ylcarbonyl)piperazin-1-yl]phenyl nonadecanoate (34)

m.p.: 61° C.

¹H-NMR (DMSO-d₆) δ=8.23 (d, 2H), 7.98 (d, 2H), 7.92 (dd, 2H), 7.7 (dd, 2H); 7.22 (dd, 1H), 6.8 (d, 1H), 6.63 (s, 1H), 6.52 (d, 1H), 4.11 (m, 2H), 3.47 (m, 2H), 3.12 (m, 2H), 2.98 (m, 2H), 1.6 (m, 6H), 1.3-1.2 (m, 31H).

### EXAMPLE 36

### 1-Hydroxy-9-[4-(3-methoxyphenyl)piperazin-1-ylcarbonyl]acridin-3-yl diphenyl phosphate (35)

¹H-NMR (DMSO-d₆) δ=11.62 (s, 1H), 8.11 (d, 1H), 7.93 (d, 1H), 7.89 (t, 1H), 7.65 (t, 1H), 7.50-7.33 (m, 11H), 7.11 (t, 1H), 6.85 (s, 1H), 6.51 (d, 1H), 6.45 (s, 1H), 6.39 (d, 1H), 4.10 (m, 1H), 3.77 (m, 1H), 3.69 (s, 3H), 3.46 (m, 1H), 3.15-3.05 (m, 4H), 2.83 (m, 1H) ppm.

### EXAMPLE 37

### 3-Hydroxy-9-[4-(3-methoxyphenyl)piperazin-1-ylcarbonyl]acridin-1-yl diphenyl phosphate (36)

¹H-NMR (DMSO-d₆) δ=10.96 (s, 1H), 8.08 (d, 1H), 7.93 (d, 1H), 7.87 (t, 1H), 7.62 (t, 1H), 7.50 (s, 1H), 7.42-7.32 (m, 11H), 7.09 (t, 1H), 6.45 (d, 1H), 6.40 (m, 2H), 3.51 (m, 4H), 3.61 (m, 2H), 3.20-3.08 (m, 4H), 2.73 (m, 1H) ppm.

### EXAMPLE 38

### (2-Hydroxyacridin-9-yl)-[4-(3-methoxyphenyl)piperazin-1-yl]methanone (37)

¹H-NMR (DMSO-d₆) δ=10.48 (br.s., 1H), 8.15 (d, 1H), 8.09 (d, 1H), 7.84 (d, 1H), 7.78 (t, 1H), 7.63 (t, 1H), 7.51 (d, 1H), 7.12 (t, 1H), 7.08 (s, 1H), 6.51 (d, 1H), 6.46 (s, 1H), 6.41 (d, 1H), 4.14 (m, 1H), 4.00 (m, 1H), 3.70 (s, 3H), 3.51 (m, 1H), 3.38 (m, 1H), 3.11 (m, 2H), 3.00 (m, 1H), 2.93 (m, 1H) ppm.

### EXAMPLE 39

### (2-Hydroxyacridin-9-yl)-[4-(3-hydroxyphenyl)piperazin-1-yl]methanone (38)

¹H-NMR (DMSO-d₆) δ=10.45 (s, 1H), 9.18 (s, 1H), 8.14 (d, 1H), 8.09 (d,1H), 7.84 (d, 1H), 7.78 (t, 1H), 7.63 (t, 1H), 7.50 (d, 1H), 7.08 (s, 1H), 7.00 (m, 1H), 6.37 (d, 1H), 6.30 (s, 1H), 6.23 (d, 1H), 4.14 (m, 1H), 3.99 (m, 1H), 3.49 (m, 2H), 3.10 (m, 2H), 2.95 (m, 1H), 2.88 (m, 1H) ppm.

### EXAMPLE 40

### (1,3-Dihydroxyacridin-9-yl)-[4-(4-methylpyridin-2-yl)piperazin-1-yl]-methanone (39)

¹H-NMR (DMSO-d₆) δ=10.94 (br.s, 1H), 10.33 (br.s, 1H), 7.97 (m, 2H), 7.83 (d, 1H), 7.76 (t, 1H), 7.47 (t, 1H), 6.85 (s, 1H), 6.66 (s, 1H), 6.53 (s, 2H), 4.03 (m, 1H), 3.84 (m, 1H), 3.72 (m, 1H), 3.63 (m, 1H), 3.53 (m, 1H), 3.15 (m, 1H), 3.04 (m, 2H), 2.20 (s, 3H) ppm.

### EXAMPLE 41

### 3-Acetoxy-9-[4-(6-methylpyridin-2-yl)piperazin-1-ylcarbonyl]acridin-1-yl acetate (40)

¹H-NMR (DMSO-d₆) δ=8.21 (d, 1H), 7.98 (d, 1H), 7.95 (t, 1H), 7.91 (s, 1H), 7.72 (dd, 1H), 7.44 (dd, 2H), 6.59 (d, 1H), 6.55 (d, 1H), 4.30 (m, 1H), 3.97 (m, 1H), 3.79 (m, 1H), 3.63 (m, 1H), 3.46 (m, 1H), 3.08-2.97 (m, 3H), 2.39 (s, 3H), 2.35 (s, 3H), 2.28 (s, 3H) ppm.

### EXAMPLE 42

### 3-Acetoxy-9-[4-(6-methoxypyridin-2-yl)piperazin-1-ylcarbonyl]acridin-1-yl acetate (41)

¹H-NMR (DMSO-d₆) δ=8.21 (d, 1H), 7.99 (d, 1H), 7.95 (t, 1H), 7.91 (s, 1H), 7.73 (t, 1H), 7.46 (m, 2H), 6.30 (d, 1H), 6.08 (d, 1 H), 4.31 (m, 1H), 3.97 (m, 1H), 3.72 (s, 3H), 3.70 (m, 1H), 3.64 (m, 1H), 3.48 (m,1 H), 3.08-2.98 (m, 3H), 2.39 (s, 3H), 2.36 (s, 3H) ppm.

The most preferred compounds of the present invention are substances of the formula I in the form of their bases or their pharmaceutically acceptable salts, which are selected from the following group:
(1,3-dihydroxyacridin-9-yl)-[4-(6-methylpyridin-2-yl)piperazin-1-yl]methanone (1)
3-[4-(acridin-9-ylcarbonyl)piperazin-1-yl]phenyl isopropylcarbamate (2)
3-[4-(acridin-9-ylcarbonyl)piperazin-1-yl]phenyl acetate (3)
mono-{3-[4-(acridin-9-ylcarbonyl)piperazin-1-yl]phenyl}phosphate (4)
3-[4-(acridin-9-ylcarbonyl)piperazin-1-yl]phenyl methyl carbonate (5)
3-[4-(acridin-9-ylcarbonyl)piperazin-1-yl]phenyl 2-chloroethyl carbonate (6)
3-[4-(acridin-9-ylcarbonyl)piperazin-1-yl]phenyl (2-hydroxyethyl)carbamate (7)
[4-(3-chlorophenyl)piperazin-1-yl]-(1,3-dihydroxyacridin-9-yl)methanone (8)
[4-(6-chloropyridin-2-yl)piperazin-1-yl]-(1,3-dihydroxyacridin-9-yl)methanone (9)
(1,3-dihydroxyacridin-9-yl)-(2,3,5,6-tetrahydro-[1,2′]-bipyrazinyl-4-yl)methanone (10)
bis-{3-[4-(acridin-9-ylcarbonyl)piperazin-1-yl]phenyl}phosphate (11)
(1,3-dihydroxyacridin-9-yl)-[4-(6-methoxypyridin-2-yl)piperazin-1-yl]methanone (12)
(1,3-dihydroxyacridin-9-yl)-[4-(3-hydroxyphenyl)piperazin-1-yl]methanone (13)
(1,3-dihydroxyacridin-9-yl)-[4-(3-methoxyphenyl)piperazin-1-yl]methanone (14)
3-[4-(acridin-9-ylcarbonyl)piperazin-1-yl]phenyl methanesulfonate (15)
3-[4-(acridin-9-ylcarbonyl)piperazin-1-yl]phenyl 2,2-dimethyl-[1,3]-dioxolan-4-ylmethyl carbonate (16)
3-(diphenoxyphosphoryloxy)-9-[4-(3-methoxyphenyl)piperazin-1-yl-carbonyl]acridin-1-yl diphenyl phosphate (17)
3-acetoxy-9-[4-(3-methoxyphenyl)piperazin-1-ylcarbonyl]acridin-1-yl acetate (18)
bis-{3-[4-(acridin-9-ylcarbonyl)piperazin-1-yl]phenyl}carbonate (19)
3-[4-(acridin-9-ylcarbonyl)piperazin-1-yl]phenyl N,N-bis-(2-hydroxyethyl)succinate (20)
3-[4-(acridin-9-ylcarbonyl)piperazin-1-yl]phenyl 4-(4-methylpiperazin-1-yl )-4-oxobutyrate (21)
mono-{3-[4-(acridin-9-ylcarbonyl)piperazin-1-yl]phenyl}glutarate (22)
3-[4-(acridin-9-ylcarbonyl)piperazin-1-y;]phenyl 5-(2,2-dimethyl-[1,3]dioxolan-4-yl)-2,2-dimethyltetrahydrofuro[2,3-d][1,3]dioxol-6-yl succinate (23)
3-[4-(acridin-9-ylcarbonyl)piperazin-1-yl]phenyl 5-[5-(2,2-dimethyl-[1,3]-dioxolan-4-yl)-2,2-dimethyltetrahydrofuro[2,3-d][1,3]dioxol-6-yloxy]-pentanoate (24)
3-[4-(acridin-9-ylcarbonyl)piperazin-1-yl]phenyl 2,3,5-trihydroxy-6-hydroxymethyltetrahydropyran-4-yl succinate (25)
3-[4-(acridin-9-ylcarbonyl)piperazin-1-yl]phenyl 5-(2,3,5-trihydroxy-6-hydroxymethyltetrahydropyran-4-yloxy)pentanoate (26)
3-[4-(acridin-9-ylcarbonyl)piperazin-1-yl]phenyl [1,4′]bispiperidinyl-1-carboxylate (27)
3-[4-(acridin-9-ylcarbonyl)piperazin-1-yl]phenyl 4-morpholin-4-yl-piperidine-1-carboxylate (28)
3-[4-(acridin-9-ylcarbonyl)piperazin-1-yl]phenyl (2-morpholin-4-ylethyl )carbamate (29)
3-[4-(acridin-9-ylcarbonyl)piperazin-1-yl]phenyl (2-chloroethyl)carbamate (30)
3-[4-(acridin-9-ylcarbonyl)piperazin-1-yl]phenyl diethylcarbamate (31)
bis(dimethylamide) mono-{3-[4-acridin-9-ylcarbonyl)piperazin-1-yl]phenyl}phosphate (32)
mono-{3-[4-(acridin-9-ylcarbonyl)piperazin-1-yl]phenyl}phosphate disodium salt (33)
3-[4-acridin-9-ylcarbonyl)piperazin-1-yl]phenyl nonadecanoate (34)
1-hydroxy-9-[4-(3-methoxyphenyl)piperazin-1-ylcarbonyl]acridin-3-yl diphenyl phosphate (35)
3-hydroxy-9-[4-(3-methoxyphenyl)piperazin-1-ylcarbonyl]acridin-1-yl diphenyl phosphate (36)
(2-hydroxyacridin-9-yl)-[4-(3-methoxyphenyl)piperazin-1-yl]methanone (37)
(2-hydroxyacridin-9-yl)-[4-(3-hydroxyphenyl)piperazin-1-yl]methanone (38)
(1,3-dihydroxyacridin-9-yl)-[4-(4-methyl pyridin-2-yl)piperazin-1-yl]methanone (39)
3-acetoxy-9-[4-(6-methyl pyridin-2-yl)piperazin-1-ylcarbonyl]acridin-1-yl acetate (40)
3-acetoxy-9-[4-(6-methoxypyridin-2-yl)piperazin-1-ylcarbonyl]acridin-1-yl acetate (41)
   Biological Actions of the Compounds According to the Invention

The in-vitro testing on selected tumor models showed the following pharmacological activities.

### EXAMPLE 43

### Antiproliferative Action on Various Tumor Cell Lines

The substances according to the invention were investigated for their antiproliferative activity in a proliferation test on established tumor cell lines. The test used determines the cellular dehydrogenase activity and makes possible a determination of the cell vitality and indirectly the cell count. The cell lines used are the human cervical carcinoma cell line KB/HeLa (ATCC CCL17), the ovarian adenocarcinoma cell line SKOV-3 (ATCC HTB77), the human glioblastoma cell line SF-68 (NCI 503138) and the lung carcinoma cell line NCI-H460 (NCI 503473). The results show a very potent inhibition of the proliferation of selected tumor cell lines by the compounds according to the invention.

TABLE 1
Inhibition of proliferation of compounds according
to the invention in the XTT cytotoxicity test on
human tumor cell lines

XTT proliferation assay, EC50 in μg/ml

| Compound | KB/Hela | SKOV3 | SF-268 | NCI-H460 |
|---|---|---|---|---|
| 2 | 0.051 | 0.039 | 0.061 | 0.062 |
| 3 | 0.013 | 0.011 | 0.018 | 0.019 |
| 4 | 0.018 | 0.208 | 0.140 | 0.272 |
| 5 | 0.017 | 0.015 | 0.024 | 0.021 |
| 6 | 0.021 | 0.019 | 0.032 | 0.029 |
| 7 | 0.029 | 0.019 | 0.031 | 0.036 |
| 11 | 0.132 | 0.133 | 0.127 | 0.293 |
| 15 | 0.078 | 0.048 | 0.069 | 0.079 |
| 16 | 0.033 | 0.026 | 0.043 | 0.042 |
| 19 | 0.020 | 0.013 | 0.025 | 0.026 |
| 20 | 0.023 | 0.014 | 0.021 | 0.027 |
| 21 | 0.024 | 0.018 | 0.027 | 0.025 |
| 22 | 0.01 | 0.007 | 0.019 | 0.016 |
| 23 | 0.045 | 0.023 | 0.058 | 0.055 |
| 24 | 0.040 | 0.023 | 0.048 | 0.072 |
| 25 | 0.019 | 0.014 | 0.029 | 0.035 |
| 26 | 0.042 | 0.026 | 0.047 | 0.054 |
| 29 | 0.036 | 0.017 | 0.024 | 0.027 |
| 30 | 0.029 | 0.019 | 0.026 | 0.025 |
| 33 | 0.013 | 0.038 | 0.057 | 0.067 |
| 37 | 0.064 | 0.042 | 0.076 | 0.067 |
| 38 | 0.125 | 0.095 | 0.137 | 0.214 |

### EXAMPLE 44

### Inhibition of the Polymerization of Tubulin

The compounds according to the invention were tested for inhibition of the polymerization of bovine tubulin in an in-vitro test. In this test, tubulin purified by cycles of polymerization and depolymerization is employed, which is polymerized by addition of GTP and warming. In Table 2, the inhibition or EC50 values of the inhibition of polymerization of tubulin with 30% associated proteins (MAPs) are indicated. The results show very potent inhibitory action of the compounds according to the invention on the polymerization of tubulin.

TABLE 2
Inhibition of tubulin polymerization. Average value from
two independent experiments. (n.d.: not determined)

Inhibition of tubulin
polymerization

| | in [% INH at 10 μg/ml] | |
|---|---|---|
| | using 30% MAPs | |
| Compound | [μg/ml] | EC50 |
| 3 | 87 | 1.35 |
| 5 | 94.3 | 1.08 |
| 6 | 70 | n.d. |
| 15 | 94.8 | 4.82 |
| 37 | n.d. | 2.06 |
| 38 | n.d. | 4.57 |

Description of the Methods Used
XTT Test for Cellular Dehydrogenase Activity

The adherently growing tumor cell lines KB/HeLa, SKOV-3, SF-268 and NCI-H460 were cultured under standard conditions in a fumigation incubator at 37° C., 5% CO₂ and 95% atmospheric humidity. On experimental day 1, the cells are detached using trypsin/EDTA and pelleted by centrifugation. Subsequently, the cell pellet is resuspended in the respective culture medium at the corresponding cell count and reacted in a 96-well microtiter plate. The plates are then cultured overnight in the fumigation incubator. The test substances are prepared as 1 mg/ml stock solutions in DMSO and diluted to the appropriate concentrations on experimental day 2 using culture medium. The substances in culture medium are then added to the cells and incubated in the fumigation incubator for 45 h. As a control, cells which are not treated with test substance are used. For the XTT assay, 1 mg/ml of XTT (sodium 3′-[1-(phenylaminocarbonyl)-3,4-tetrazolium]-bis(4-methoxy-6-nitro)benzenesulfonic acid) is dissolved in RPMI-1640 medium without Phenol Red. Additionally, a 0.383 mg/ml PMS (N-methyldibenzopyrazine methylsulfate) solution in phosphate-buffered saline solution (PBS) is prepared. On experimental day 4, 75 μl/well of XTT-PMS mixture is pipetted onto the cell plates which in the meantime have been incubated with the test substances for 45 h. For this, shortly before use, the XTT solution is mixed with the PMS solution in the ratio 50:1 (vol:vol). The cell plates are then incubated in the fumigation incubator for a further 3 h and the optical density (OD_{490 nm}) is determined in a photometer. By means of the OD_{490 nm} determined, the percentage inhibition is calculated relative to the control and plotted semilogarithmically in the form of a concentration-action curve. The EC₅₀ is calculated by means of a regression analysis from the concentration-action curve using the program Graphpad Prism.

Tubulin Polymerization Assay

The assay is carried out based on the method of Bollag et al. Lyophilized bovine tubulin (cytoskeleton, ML113 tubulin 30% MAPs) is dissolved in a concentration of 2 mg/ml (ML113 in 80 mM PIPES, 0.5 mM EGTA, 2 mM MgCl₂, pH 6.9, 1 mM GTP) or 5 mg/ml (TL238 in 80 mM PIPES, 1 mM EGTA, 0.5 mM MgCl₂, 20% (v:v) glycerol pH 6.9, 1 mM GTP). The test substances are diluted in 10% DMSO (v:v) and 5 μl of the dilutions are transferred to a 96-well microtiter plate (Nunc, half area plate). After addition of 45 μl of the tubulin solution, the polymerization is determined at 340 nm in a Spectramax 190 microtiter plate reader (Molecular Devices) by means of a kinetics program at 30 sec intervals over a period of 20 min. The resulting area under curve values are used for the calculation of the inhibition with respect to the untreated control. The controls are untreated cells (±induction). Induction was carried out using 3 μM of muristerone A. On day 1, the cells are sown (±muristerone A) and incubated at 37° C. for 24 h. On day 2, the test substance is added (control DMSO) and incubation at 37° C. is continued further 45 h, after which a standard XTT assay is carried out.

### EXAMPLE 45

### Saturation Solubility in Water

The saturation solubility in water is determined as described below. For incipient dissolution of the substances and to improve wetting of the samples, at most 1% of DMSO is added. To check the content, an HPLC-UV method was used. The results are summarized in the table below.

| | Absolute solubility in |
|---|---|
| Compound | water [μg/ml] |
| 1 | 83.9 |
| 4 | 123 |
| 7 | 108.3 |
| 20 | 143.5 |
| 21 | 114.8 |
| 25 | 215.3 |
| 29 | 41.03 |
| 33 | 9085.8 |
| Acridin-9-yl-[4-(3-methoxyphenyl)piperazin-1- | 0.42 |
| yl]-methanone (WO0208194) | |
| Acridin-9-yl-[4-(6-methylpyridin-2-yl)piperazin- | 1.07 |
| 1-yl]methanone (WO0208194) | |
| Acridin-9-yl-[4-(3-hydroxyphenyl)piperazin-1- | 0.03 |
| yl]methanone (WO0208194) | |

The compounds 1, 4, 7, 20, 21, 25, 29 and 33 have greatly improved water solubilities compared to the comparison substances.

Examples of Pharmaceutical Administration Forms

### EXAMPLE I

Tablet containing 50 mg of active compound

| Composition: | |
|---|---|
| (1) Active compound | 50.0 mg |
| (2) Lactose | 98.0 mg |
| (3) Cornstarch | 50.0 mg |
| (4) Polyvinylpyrrolidone | 15.0 mg |
| (5) Magnesium stearate | 2.0 mg |
| Total: | 215.0 mg |

Preparation:
(1), (2) and (3) are mixed and granulated with an aqueous solution of (4). (5) is admixed to the dried granules. Tablets are pressed from this mixture.

### EXAMPLE II

Capsule containing 50 mg of active compound

| Composition: | |
|---|---|
| (1) Active compound | 50.0 mg |
| (2) Cornstarch, dried | 58.0 mg |
| (3) Lactose, powdered | 50.0 mg |
| (4) Magnesium stearate | 2.0 mg |
| Total: | 160.0 mg |

Preparation:
(1) is triturated with (3). This trituration is added to the mixture of (2) and (4) with intensive mixing. This powder mixture is filled into hard gelatine capsules size 3 on a capsule filling machine.

1. An acridine derivative of the formula 1 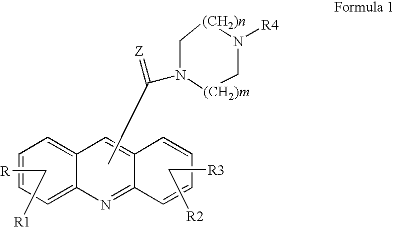
where 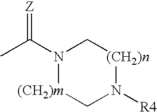
may be attached to carbon atoms C₁ to C₉ of the ring skeleton;
Z: is oxygen or sulfur;
n,m: independently of one another are integers from 0 to 4;
R, R1, R2, R3: may optionally be attached to the heteroaromatic carbon atoms C₁ to C₉ of the acridine, are identical or different and independently of one another are hydrogen, hydroxyl or OR5, but the radicals R, R1, R2 and R3 are not simultaneously hydrogen,
R4: is a (C₆-C₁₄)-aryl radical, a (C₆-C₁₄)-aryl-(C₁-C₄)-alkyl radical or a (C₂-C₁₀)-heteroaryl or (C₂-C₁₀)-heteroaryl-(C₁-C₄)-alkyl radical containing one or more heteroatoms selected from the group consisting of N, O and S, where the (C₁-C₄)-alkyl radical may be unsubstituted or mono- or polysubstituted by identical or different substituents from the group consisting of (C₁-C₆)-alkyl and halogen and the (C₆-C₁₄)-aryl or (C₂-C₁₀)-heteroaryl radical may be unsubstituted or mono- or polysubstituted by identical or different substituents from the group consisting of straight-chain or branched (C₁-C₈)-alkyl, (C₃-C₁₂)-cycloalkyl, straight-chain or branched (C₁-C₈)-alkylcarbonyl, hydroxyl, straight-chain or branched (C₁-C₈)-alkoxy, OR5, halogen, straight-chain or branched aryl-(C₁-C₈)-alkoxy, trityloxy, trimethylsilyloxy, amino, mono-(C₁-C₄)-alkylamino, di-(C₁-C₄)-alkylamino, (C₂-C₅)-cycloalkylamino, morpholino, heterocyclyl-(C₁-C₆)-alkoxy, carboxyl, imidocarboxyl, carboxamidine, straight-chain or branched (C₁-C₈)-alkoxycarbonylamino, straight-chain or branched (C₁-C₈)-alkylcarbonylamino, sulfonyloxy, sulfenyloxy, sulfinyloxy, nitro, nitroso, thiol, straight-chain or branched (C₁-C₈)-alkylthio, straight-chain or branched (C₁-C₈)-alkylsulfonyl, straight-chain or branched (C₁-C₈)-alkylsulfoxy, cyano, isocyanato, straight-chain or branched cyano-(C₁-C₆)-alkyl, straight-chain or branched (C₁-C₈)-alkoxycarbonyl, straight-chain or branched (C₁-C₄)-alkyl which is substituted by one or more halogen atoms, straight-chain or branched carboxy-(C₁-C₈)-alkyl, straight-chain or branched (C₁-C₈)-alkoxycarbonyl-(C₁-C₆)-alkyl, (C₂-C₆)-alkenyl and (C₂-C₆)-alkynyl,
or, if R, R1, R2, R3 may optionally be attached to the heteroaromatic carbon atoms C₁ to C₉ of the acridine, are identical or different and independently of one another:
are hydrogen, straight-chain or branched (C₁-C₈)-alkyl, (C₃-C₇)-cycloalkyl, straight-chain or branched (C₁-C₈)-alkylcarbonyl, hydroxyl, straight-chain or branched (C₁-C₈)-alkoxy, halogen, straight-chain or branched aryl-(C₁-C₈)-alkoxy, trityloxy, trimethylsilyloxy, amino, mono-(C₁-C₄)-alkylamino, di-(C₁-C₄)-alkylamino, (C₂-C₅)-cycloalkylamino, morpholino, heterocyclyl-(C₁-C₆)-alkoxy, carboxyl, imidocarboxyl, carboxamidine, straight-chain or branched (C₁-C₈)-alkoxycarbonylamino, straight-chain or branched (C₁-C₈)-alkylcarbonylamino, sulfonyloxy, sulfenyloxy, sulfinyloxy, nitro, nitroso, thiol, straight-chain or branched (C₁-C₈)-alkylthio, straight-chain or branched (C₁-C₈)-alkylsulfonyl, straight-chain or branched (C₁-C₈)-alkylsulfoxy, cyano, isocyanato, straight-chain or branched cyano-(C₁-C₆)-alkyl, straight-chain or branched (C₁-C₈)-alkoxycarbonyl, straight-chain or branched (C₁-C₄)-alkyl which is substituted by one or more halogen atoms, straight-chain or branched carboxy-(C₁-C₈)-alkyl, straight-chain or branched (C₁-C₈)-alkoxycarbonyl-(C₁-C₆)-alkyl, (C₂-C₆)-alkenyl, (C₂-C₆)-alkynyl, aryl, where the aryl radical may be unsubstituted or mono- or polysubstituted by identical or different substituents from the group consisting of halogen, straight-chain or branched (C₁-C₈)-alkyl, (C₃-C₁₂)-cycloalkyl, straight-chain or branched (C₁-C₈)-alkylcarbonyl, hydroxyl, straight-chain or branched (C₁-C₈)-alkoxy, amino, mono-(C₁-C₄)-alkylamino, di-(C₁-C₄)-alkylamino, (C₂-C₅)-cycloalkylamino, morpholino, heterocyclyl-(C₁-C₆)-alkoxy, carboxyl, imidocarboxyl, carboxamidine, straight-chain or branched (C₁-C₈)-alkoxycarbonylamino, straight-chain or branched (C₁-C₈)-alkylcarbonylamino, sulfonyloxy, sulfenyloxy, sulfinyloxy, nitro, nitroso, thiol, straight-chain or branched (C₁-C₈)-alkylthio, cyano, isocyanato, straight-chain or branched (C₁-C₈)-alkoxycarbonyl, straight-chain or branched (C₁-C₄)-alkyl, (C₂-C₆)-alkenyl, (C₂-C₆)-alkynyl, substituted by one or more halogen atoms,
R4 is: a (C₆-C₁₄)-aryl radical, a (C₆-C₁₄)-aryl-(C₁-C₄)-alkyl radical or a (C₂-C₁₀)-heteroaryl or (C₂-C₁₀)-heteroaryl-(C₁-C₄)-alkyl radical which contains one or more heteroatoms selected from the group consisting of N, O and S, where the (C₁-C₄)-alkyl radical may be unsubstituted or mono- or polysubstituted by identical or different substituents from the group consisting of (C₁-C₆)-alkyl and halogen and the (C₆-C₁₄)-aryl or (C₂-C₁₀)-heteroaryl radical may be mono- or polysubstituted by identical or different OR5,
and in all cases, R5 may be:
a sulfone of the formula —SO2-X1, where X1 is NMe₂, hydroxyl, O-alkyl, unsubstituted or substituted alkyl, unsubstituted or substituted cycloalkyl, unsubstituted or substituted aryl, unsubstituted or substituted heteroaryl, unsubstituted or substituted alkylcycloalkyl, unsubstituted or substituted alkylheterocyclyl, unsubstituted or substituted alkylaryl, unsubstituted or substituted alkylheteroaryl;
—C(O)—X2, where X2 is unsubstituted or substituted alkyl, unsubstituted or substituted cycloalkyl, unsubstituted or substituted heterocyclyl, unsubstituted or substituted aryl, unsubstituted or substituted heteroaryl, unsubstituted or substituted alkylcycloalkyl, unsubstituted or substituted alkylheterocyclyl, unsubstituted or substituted alkylaryl or unsubstituted or substituted alkylheteroaryl,
—C(O)O—X3, where X3 is unsubstituted or substituted alkyl, unsubstituted or substituted cycloalkyl, unsubstituted or substituted heterocyclyl, unsubstituted or substituted aryl, unsubstituted or substituted heteroaryl, unsubstituted or substituted alkylcycloalkyl, unsubstituted or substituted alkylheterocyclyl, unsubstituted or substituted alkylaryl or unsubstituted or substituted alkylheteroaryl,
—C(O)NX4X5, where X4 and X5 independently of one another are hydrogen, unsubstituted or substituted alkyl, unsubstituted or substituted cycloalkyl, unsubstituted or substituted heterocyclyl, unsubstituted or substituted aryl, unsubstituted or substituted heteroaryl, unsubstituted or substituted alkylcycloalkyl, unsubstituted or substituted alkylheterocyclyl, unsubstituted or substituted alkylaryl, unsubstituted or substituted alkylheteroaryl, or X4 and X5 together are cycloalkyl or cycloheteroalkyl,
—P(O)OX6OX7, where X6 and X7 independently of one another are hydrogen, a metal, unsubstituted or substituted alkyl, unsubstituted or substituted cycloalkyl, unsubstituted or substituted heterocyclyl, unsubstituted or substituted aryl, unsubstituted or substituted heteroaryl, unsubstituted or substituted alkylcycloalkyl, unsubstituted or substituted alkylheterocyclyl, unsubstituted or substituted alkylaryl, unsubstituted or substituted alkylheteroaryl, or X6 and X7 together are cycloalkyl or cycloheteroalkyl,
—P(O)NX8X9NX10X11, where X8, X9, X10 and X11 independently of one another are hydrogen, unsubstituted or substituted alkyl, unsubstituted or substituted cycloalkyl, unsubstituted or substituted heterocyclyl, unsubstituted or substituted aryl, unsubstituted or substituted heteroaryl, unsubstituted or substituted alkylcycloalkyl, unsubstituted or substituted alkylheterocyclyl, unsubstituted or substituted alkylaryl, unsubstituted or substituted alkylheteroaryl, or X8 and X9 or X10 and X11 together are cycloalkyl or cycloheteroalkyl,
cycloalkyl, alkylcycloalkyl, cycloheteroalkyl or alkylcycloheteroalkyl;
or a physiologically acceptable salt thereof.

2. An acridine derivative of the formula 1 as claimed in claim 1, wherein the alkyl radical may be methyl, ethyl, n-propyl, 2-propyl, n-butyl, sec-butyl, tert-butyl, n-pentyl, iso-pentyl, neo-pentyl, n-hexyl, 2-hexyl, n-octyl, ethylenyl (vinyl), ethynyl, propenyl (—CH₂CH═CH₂; —CH═CH—CH₃, —C(═CH₂)—CH₃), propynyl (—CH₂—C≡CH, —C≡C—CH₃), butenyl, butynyl, pentenyl, pentynyl, hexenyl, hexynyl, octenyl or octynyl.

3. An acridine derivative of the formula 1 as claimed in claim 1, wherein the heterocyclyl radical may be tetrahydrofuryl, tetrahydropyranyl, pyrrolidinyl, piperidinyl, Piperazinyl or morpholinyl.

4. An acridine derivative of the formula 1 as claimed in claim 1, wherein the heteroaryl radical may be pyrrolyl, furyl, thienyl, thiazolyl, triazolyl, tetrazolyl, oxazolyl, isothiazolyl, isoxazolyl, pyrazolyl, imidazolyl, pyridinyl, pyrimidinyl, pyrazinyl, triazinyl, benzothiazolyl, indolyl, indolizinyl, quinolinyl, isoquinolinyl, cinnolinyl, quinazolinyl, quinoxalinyl, phthalazinyl, carbazolyl, phenazinyl, phenothiazinyl, purinyl, acridinyl or phenanthrinyl.

5. An acridine derivative of the formula 1 as claimed in claim 1, wherein the physiologically acceptable salt is formed by neutralization of the base with an inorganic or organic acid or neutralization of the acid with an inorganic or organic base.

6. An acridine derivative of the formula 1 as claimed in claim 1 having at least one asymmetric carbon atom, in the form of its racemate, in the form of an enantiomer and/or diastereoisomer or in the form of mixtures of these enantiomers and/or diastereoisomers, in the form of a tautomer, solvate and hydrate and polymorph thereof.

7. An acridine derivative of the formula 1 as claimed in claim 1, which is one of the following compounds:
(1,3-dihydroxyacridin-9-yl)-[4-(6-methyl pyridin-2-yl )piperazin-1-yl]methanone (1)
3-[4-(acridin-9-ylcarbonyl)piperazin-1-yl]phenyl isopropylcarbamate (2)
3-[4-(acridin-9-ylcarbonyl)piperazin-1-yl]phenyl acetate (3)
mono-{3-[4-(acridin-9-ylcarbonyl)piperazin-1-yl]phenyl}phosphate (4)
3-[4-(acridin-9-ylcarbonyl)piperazin-1-yl]phenyl methyl carbonate (5)
3-[4-(acridin-9-ylcarbonyl)piperazin-1-yl]phenyl 2-chloroethyl carbonate (6)
3-[4-(acridin-9-ylcarbonyl)piperazin-1-yl]phenyl (2-hydroxyethyl)carbamate (7)
[4-(3-chlorophenyl)piperazin-1-yl]-(1,3-dihydroxyacridin-9-yl)methanone (8)
[4-(6-chloropyridin-2-yl)piperazin-1-yl]-(1,3-dihydroxyacridin-9-yl)methanone (9)
(1,3-dihydroxyacridin-9-yl)-(2,3,5,6-tetrahydro-[1,2′]-bipyrazinyl-4-yl)methanone (10)
bis-{3-[4-(acridin-9-ylcarbonyl)piperazin-1-yl]phenyl}phosphate (11)
(1,3-dihydroxyacridin-9-yl)-[4-(6-methoxypyridin-2-yl)piperazin-1-yl]methanone (12)
(1,3-dihydroxyacridin-9-yl)-[4-(3-hydroxyphenyl)piperazin-1-yl]methanone (13)
(1,3-dihydroxyacridin-9-yl )-[4-(3-methoxyphenyl)piperazin-1-yl]methanone (14)
3-[4-(acridin-9-ylcarbonyl)piperazin-1-yl]phenyl methanesulfonate (15)
3-[4-(acridin-9-ylcarbonyl)piperazin-1-yl]phenyl 2,2-dimethyl-[1,3]-dioxolan-4-ylmethyl carbonate (16)
3-(diphenoxyphosphoryloxy)-9-[4-(3-methoxyphenyl )piperazin-1-yl-carbonyl]acridin-1-yl diphenyl phosphate (17)
3-acetoxy-9-[4-(3-methoxyphenyl)piperazin-1-ylcarbonyl]acridin-1-yl acetate (18)
bis-{3-[4-(acridin-9-ylcarbonyl)piperazin-1-yl]phenyl}carbonate (19)
3-[4-(acridin-9-ylcarbonyl)piperazin-1-yl]phenyl N,N-bis-(2-hydroxyethyl)succinate (20)
3-[4-(acridin-9-ylcarbonyl)piperazin-1-yl]phenyl 4-(4-methyl piperazin-1-yl )-4-oxobutyrate (21)
mono-{3-[4-(acridin-9-ylcarbonyl)piperazin-1-yl]phenyl}glutarate (22)
3-[4-(acridin-9-ylcarbonyl)piperazin-1-yl]phenyl 5-(2,2-dimethyl-[1,3]dioxolan-4-yl)-2,2-dimethyltetrahydrofuro[2,3-d][1,3]dioxol-6-yl succinate (23)
3-[4-(acridin-9-ylcarbonyl)piperazin-1-yl]phenyl 5-[5-(2,2-dimethyl-[1,3]-dioxolan-4-yl)-2,2-dimethyltetrahydrofuro[2,3-d][1,3]dioxol-6-yloxy]-pentanoate (24)
3-[4-(acridin-9-ylcarbonyl)piperazin-1-yl]phenyl 2,3,5-trihydroxy-6-hydroxymethyltetrahydropyran-4-yl succinate (25)
3-[4-(acridin-9-ylcarbonyl)piperazin-1-yl]phenyl 5-(2,3,5-trihydroxy-6-hydroxymethyltetrahydropyran-4-yloxy)pentanoate (26)
3-[4-(acridin-9-ylcarbonyl)piperazin-1-yl]phenyl[1,4′]bispiperidinyl-1′-carboxylate (27)
3-[4-(acridin-9-ylcarbonyl)piperazin-1-yl]phenyl 4-morpholin-4-yl-piperidine-1-carboxylate (28)
3-[4-(acridin-9-ylcarbonyl)piperazin-1-yl]phenyl (2-morpholin-4-ylethyl)carbamate (29)
3-[4-(acridin-9-ylcarbonyl)piperazin-1-yl]phenyl (2-chloroethyl)carbamate (30)
3-[4-(acridin-9-ylcarbonyl)piperazin-1-yl]phenyl diethylcarbamate (31)
bis(dimethylamide) mono-{3-[4-acridin-9-ylcarbonyl)piperazin-1-yl]phenyl}phosphate (32)
mono-{3-[4-(acridin-9-ylcarbonyl)piperazin-1-yl]phenyl}phosphate disodium salt (33)
3-[4-acridin-9-ylcarbonyl)piperazin-1-yl]phenyl nonadecanoate (34)
1-hydroxy-9-[4-(3-methoxyphenyl)piperazin-1-ylcarbonyl]acridin-3-yl diphenyl phosphate (35)
3-hydroxy-9-[4-(3-methoxyphenyl)piperazin-1-ylcarbonyl]acridin-1-yl diphenyl phosphate (36)
(2-hydroxyacridin-9-yl)-[4-(3-methoxyphenyl)piperazin-1-yl]methanone (37)
(2-hydroxyacridin-9-yl)-[4-(3-hydroxyphenyl)piperazin-1-yl]methanone (38)
(1,3-dihydroxyacridin-9-yl)-[4-(4-methylpyridin-2-yl)piperazin-1-yl]methanone (39)
3-acetoxy-9-[4-(6-methylpyridin-2-yl)piperazin-1-ylcarbonyl]acridin-1-yl acetate (40)
3-acetoxy-9-[4-(6-methoxypyridin-2-yl)piperazin-1-ylcarbonyl]acridin-1-yl acetate (41).

8. A pharmaceutical composition comprising an acridine derivative of the formula 1 as claimed in claim 1.

9. A method for treating a tumor in a human or mammal comprising administering an acridine derivative of the formula 1 as claimed in claim 1 to the human or mammal.

10. A process for preparing acridine derivatives of the formula 1 as claimed in claim 1, which comprises reacting an acridine carboxylic acid of the formula 2 in which R, R1, R2, R3 are as defined in claim 1, Z is an oxygen or sulfur atom and Y is a leaving group, 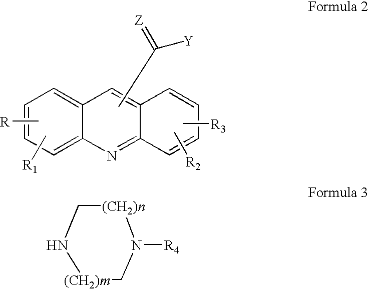
with an amine of the formula 3 in which R₄, m and n are as defined in claim 1, using, if appropriate, a condensing agent and/or a catalyst and a diluent and auxiliary, with formation of the desired acridine derivative.

11. A pharmaceutical composition comprising at least one acridine derivative of the formula 1 as claimed in claim 1 and a pharmaceutically acceptable excipient, additive or carrier.

12. A method for treating benign and malignant tumors in a human or mammal, which comprises administering at least one acridine derivative of the formula 1 as claimed in claim 1 to the human or mammal, in a dose effective for tumor treatment.

13. A process of claim 10, wherein the leaving group is halogen, hydroxyl, (C₁-C₆)-alkoxy, —O-tosyl, —O-mesyl, tetrazolyl or imidazolyl.

14. A process of claim 13, wherein in the leaving group is methoxy or ethoxy.
